(19) United States
(12) Patent Application Publication
(10) Pub. No.: US 2021/0033543 A1
(43) Pub. Date: Feb. 4, 2021

(54) RADIATION GENERATION CONTROL DEVICE, RADIATION GENERATION CONTROL SYSTEM, AND RADIOGRAPHY SYSTEM

(71) Applicant: Konica Minolta, Inc., Tokyo (JP)

(72) Inventor: Masahiro KUWATA, Machida-shi (JP)

(21) Appl. No.: 16/911,031

(22) Filed: Jun. 24, 2020

(30) Foreign Application Priority Data
Aug. 2, 2019 (JP) 2019-142716

(51) Int. Cl. G01N 23/04

(52) U.S. Cl. CPC G01N 23/04; A61B 6/58

(57) ABSTRACT

A radiation generation control device includes an acquisitor that acquires a first signal that instructs emission of radiation, a first connector that inputs a second signal indicating a driving state of a radiation image capturing device that generates a radiation image, a second connector connectable to a radiation generation device that generates radiation, and a controller that, based on the first signal having been acquired and the second signal having been input, causes a third signal that instructs emission of radiation to be output repeatedly from the second connector in a predetermined cycle.

### BACKGROUND

### 1. Technological Field

The present invention relates to a radiation generation control device, a radiation generation control system, and a radiography system.

### 2. Description of the Related Art

Conventionally, technologies for fluoroscopically seeing the inside of a subject using radiation have generally been divided into those for capturing low-quality moving images using a camera and those for capturing high-quality still images using a film or fluorescent plate.

Examples of a device for capturing moving images include a radiation video device as described in JP 09-270955A including a TV camera that generates a radiographic image and a radiation high-voltage device that applies a pulsed high voltage synchronized with an image acquiring operation performed by the TV camera to a radiation tube while an exposure switch is being pressed.

In the field of still image capturing, a radiation image capturing device (flat panel detector) has been newly developed which includes a substrate on which a plurality of pixels are arrayed two-dimensionally, and reads out, as image data, the amount of charges generated in each pixel in accordance with the intensity of radiation emitted from a radiation generation device through a subject to perform still image capturing.

Therefore, as described in JP 6039225B, for example, a radiography system in which such a radiography device is used instead of a film or fluorescent plate has been proposed.

In recent years, radiography devices have been improved further in performance, and there are some radiography devices having an image capturing capacity for repeatedly performing still image capturing multiple times for a short time. Therefore, an attempt has been made to continue emitting radiation to this radiography device for a predetermined period, and meanwhile cause generation of a still image to be repeated to capture the kinetic state of an examination target area of a subject in the form of a plurality of sequential still images and to apply the still images to diagnoses. Hereinafter, such image capturing of repeating generation of still images for a short time will be referred to as kymography.

However, in a conventional radiography system as described in JP 6039225B, even though the radiography device can be replaced with a device adaptable to kymography as described above, the radiation generation device emits pulsed radiation only once in response to an instruction to emit radiation once, or emits radiation for a period in which the exposure switch is being pressed, so that kymography in which the number of captured images is strictly managed is not performed appropriately.

### SUMMARY

The present invention was made in view of the above-described points, and has an object to easily alter an existing radiation generation device that emits pulsed radiation only once in response to an instruction to emit radiation once or emits radiation for a period in which a user is performing a predetermined manipulation to a device adaptable to kymography.

In order to solve the aforementioned problems, a radiation generation control device according to an aspect of the present invention includes:

an acquisitor that acquires a first signal that instructs emission of radiation;

a first connector that inputs a second signal indicating a driving state of a radiation image capturing device that generates a radiation image;

a second connector connectable to a radiation generation device that generates radiation; and

a controller that, based on the first signal having been acquired and the second signal having been input, continuously causes a third signal that instructs emission of radiation to be output from the second connector for a predetermined period, wherein

the controller determines the length of the predetermined period in accordance with an image capturing time or the number of captured images previously set.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention.

FIG. 1 is a block diagram representing a radiography system according to Conventional technology 1.

FIG. 2 is a block diagram representing a radiography system according to a first embodiment (second embodiment) of the present invention.

FIG. 3 is a block diagram of a radiation image capturing device included in the radiography system of FIG. 2.

FIG. 4 is a ladder chart representing the first half of operations of the radiography system according to the first embodiment.

FIG. 5 is a ladder chart representing the latter half of the operations of the radiography system according to the first embodiment.

FIG. 6 is a timing chart representing the operations of the radiography system of FIG. 2.

FIG. 7 is a ladder chart representing the first half of operations of the radiography system according to the second embodiment of the present invention.

FIG. 8 is a ladder chart representing the latter half of the operations of the radiography system according to the second embodiment.

FIG. 9 is a block diagram representing a radiography system according to Conventional technology 2.

FIG. 10 is a block diagram representing a radiography system according to a third embodiment (fourth embodiment) of the present invention.

FIG. 11 is a ladder chart representing the first half of operations of the radiography system according to the third embodiment.

FIG. 12 is a ladder chart representing the latter half of the operations of the radiography system according to the third embodiment.

FIG. 13 is a ladder chart representing the first half of operations of the radiography system according to the fourth embodiment of the present invention.

FIG. 14 is a ladder chart representing the latter half of the operations of the radiography system according to the fourth embodiment.

FIG. 15 is a state transition diagram describing a transition of a state of the radiography system of FIG. 2 or FIG. 10.

FIG. 16 is a timing chart representing the operation of the radiography systems according to the first and third embodiments.

FIG. 17 is a timing chart representing the operation of the radiography systems according to the second and fourth embodiments.

FIG. 18 is a block diagram representing another configuration example of the radiography system of FIG. 2 or FIG. 10.

FIG. 19 is an example of a display screen of a display of a console included in the radiography system of FIG. 2 or FIG. 10.

FIG. 20 is a block diagram representing another configuration example of the radiography system of FIG. 2 or FIG. 10.

FIG. 21 is an example of the display screen of the display of the console included in the radiography system of FIG. 2 or FIG. 10.

FIG. 22 is a timing chart representing the operations of the radiography system of FIG. 2 or FIG. 10.

FIG. 23 is a timing chart representing the operations of the radiography system of FIG. 2 or FIG. 10.

FIG. 24 is a ladder chart representing the latter half of the operations of the radiography system of the second embodiment.

FIG. 25 is a ladder chart representing the latter half of the operations of the radiography system of the fourth embodiment.

FIG. 26 shows an example of an apparatus configuration and a connection configuration of a radiography system displayed on a display of a console.

### DETAILED DESCRIPTION OF THE EMBODIMENTS

Hereinafter, embodiments of the present invention and conventional technologies on which the embodiments are based will be described with reference to the drawings. However, the technical scope of the present invention is not limited to the description of the following embodiments or illustration in the drawings.

Herein, Conventional technology 1 on which first and second embodiments are based will be described, and the first embodiment and the second embodiment will be described sequentially. Then, Conventional technology 2 on which third and fourth embodiments are based will be described, and the third embodiment and the fourth embodiment will be described sequentially

Conventional Technology 1

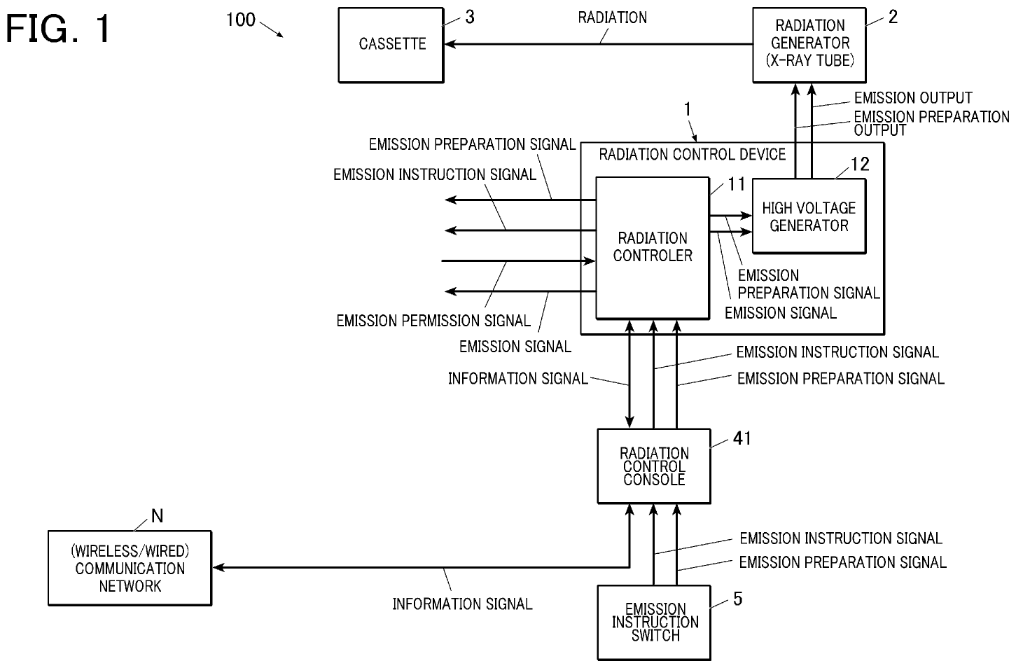

First, Conventional technology 1 on which radiography systems (details of which will be described later) according to the first and second embodiments of the present invention are based will be described with reference to FIG. 1.

System Configuration

First, a schematic configuration of the radiography system according to Conventional technology 1 (hereinafter, a conventional system 100) will be described. FIG. 1 is a block diagram representing the conventional system 100.

The conventional system 100 includes a radiation controller 11, a high voltage generator 12, a radiation generator 2, a cassette 3, a radiation control console 41, and an emission instruction switch 5 as shown in FIG. 1, for example, and performs still image capturing through use of a radiographic film, CR, or the like, in which a radiation emission timing and an image capturing timing do not interact with each other.

FIG. 1 illustrates a case in which the radiation controller 11 and the high voltage generator 12 both constitute the radiation control device 1 (for example, they are stored in a single enclosure), whilst the radiation controller 11 and the high voltage generator 12 may be configured independently from each other by being arranged in different enclosures, or the like, for example.

The radiation controller 11, the high voltage generator 12, and the radiation generator 2 constitute the radiation generation device in the present invention.

The radiation controller 11 is to control radiation emission.

Specifically, upon sensing that an emission preparation signal from the radiation control console 41 has been turned on, the radiation controller 11 turns on the emission preparation signal to be output to the high voltage generator 12 or brings the emission preparation signal into a state allowed to be output to another external apparatus.

Upon sensing that an emission instruction signal (a first signal in the present invention) that instructs emission of radiation from the radiation control console 41 has been turned on, the radiation controller 11 brings this emission instruction signal into a state allowed to be output to an external apparatus, and transmits an emission signal in accordance with image capturing conditions set by the radiation control console 41 to the high voltage generator 12.

These emission preparation signal and emission instruction signal allowed to be output from the radiation controller 11 to an external apparatus are used in a case where the external apparatus is connected to the radiation controller 11, for example.

By virtue of these emission preparation signal and emission instruction signal, in image capturing that requires preparation for an external apparatus other than the cassette 3 when radiation is emitted, the external apparatus performs preparation for image capturing based on the emission preparation signal and emission instruction signal output from the radiation controller 11.

Examples of such an external apparatus include a grid swinging device provided on a radiation incident surface of the cassette 3 and used to swing a grid when capturing an image, and the like.

The above-described external apparatus includes an apparatus that transmits an emission permission signal to the radiation controller 11 after preparation for image capturing is completed. Thus, the radiation controller 11 may include a connector to which the emission permission signal is to be input from an external apparatus so as to transmit the emission signal to the high voltage generator 12 only in a case where both the emission instruction signal from the radiation control console 41 and the emission permission signal from the external apparatus are turned on.

Accordingly, the emission permission signal is not input to the radiation controller 11 before preparation for image capturing in the external apparatus is completed, so that radiation is prevented from being emitted before preparation for image capturing in the external apparatus is completed.

For example, in a case where the external apparatus is the aforementioned grid swinging device, the grid swinging device inputs an emission permission signal from the grid swinging device to the radiation controller 11 after the grid swinging device starts swinging and a designated swinging speed is attained. Accordingly, the radiation controller 11 outputs an emission signal only after both the emission instruction signal from the emission instruction switch 5 based on a manipulation of a radiographer and the emission permission signal from the external apparatus are received, so that radiation is prevented from being emitted before preparation in the external apparatus is completed.

In a case where it is not desired to use the emission permission signal from the external apparatus in the radiation controller 11, the emission permission signal needs to be disabled, or the emission permission signal needs to be maintained in the on or off state all the time, for example.

For example, in a case where the radiation controller 11 selects whether to use the emission permission signal from the external apparatus for determining whether to permit output of the emission signal, the emission permission signal is disabled by selecting not to be used for the determination.

In a case where such a selection is not allowed, and in a case where the emission permission signal is instructed by opening or closing two signal lines, for example, the emission permission signal is maintained in the on or off state all the time by making the two signal lines open or closed all the time.

The radiation controller 11 may be configured not to transmit the emission signal even upon sensing that the emission instruction signal has been turned on, until a predetermined waiting time elapses upon sensing that the emission preparation signal has been turned on.

Accordingly, in a case where the high voltage generator 12 and the radiation generator require time for preparation to some degree upon sensing that the emission preparation signal has been turned on, radiation is prevented from being emitted even though emission preparation has not been completed.

Upon sensing that the emission preparation signal from the radiation controller 11 has been turned on, the high voltage generator 12 outputs an emission preparation output to the radiation generator 2.

Upon receipt of the emission signal from the radiation controller 11, the high voltage generator 12 applies a high voltage necessary for the radiation generator 2 to generate radiation (in accordance with the input emission signal) to the radiation generator 2 as an emission output.

FIG. 1 illustrates the configuration in which, when the high voltage generator 12 senses that the emission preparation signal from the radiation controller 11 has been turned on, the high voltage generator 12 performs an emission preparation output to the radiation generator 2, whilst the radiation controller 11 may directly output the emission preparation signal to the radiation generator 2 for conversion into the emission preparation output in the radiation generator 2 to perform emission preparation.

The radiation generator 2 (x-ray tube) includes an electron gun and an anode, for example, and generates radiation (for example, X-rays) in accordance with the high voltage applied from the high voltage generator 12.

Specifically, when the high voltage is applied, the electron gun emits an electron beam to the anode, and the anode generates radiation upon receipt of the electron beam.

Since the anode when generating radiation produces heat at a portion having received the electron beam to be raised in temperature, the position on the anode at which an electron beam is emitted needs to be changed continually in order for stable radiation emission. Therefore, a rotary anode that emits an electron beam while rotating the anode may be used.

The emission preparation output from the above-described high voltage generator 12 is used as an instruction for the start of rotation of the rotary anode, for example.

It is considered that the radiation generation device (the radiation controller 11, the high voltage generator 12, and the radiation generator 2) configured in this manner operates in some cases in a mode of emitting pulsed radiation immediately after the emission instruction signal and the emission permission signal are turned on (hereinafter, a pulse emission mode), and operates in other cases in a mode of continuing emitting radiation for a period in which the emission instruction signal and the emission permission signal are maintained in the on state (hereinafter, a continuous emission mode).

Depending on the types of the radiation controller 11, the high voltage generator 12, and the radiation generator 2, the radiation generation device may be operable only in either the pulse emission mode or the continuous emission mode, or may be adaptable to both the modes.

The cassette 3 stores a radiology film or fluorescent plate, and when radiation passed through a subject enters, forms a radiation image of the subject.

The radiation control console 41 uses an information signal connection to set subject-related information and image capturing conditions (a tube voltage, a tube current, an emission time, and the like) in the radiation controller 11.

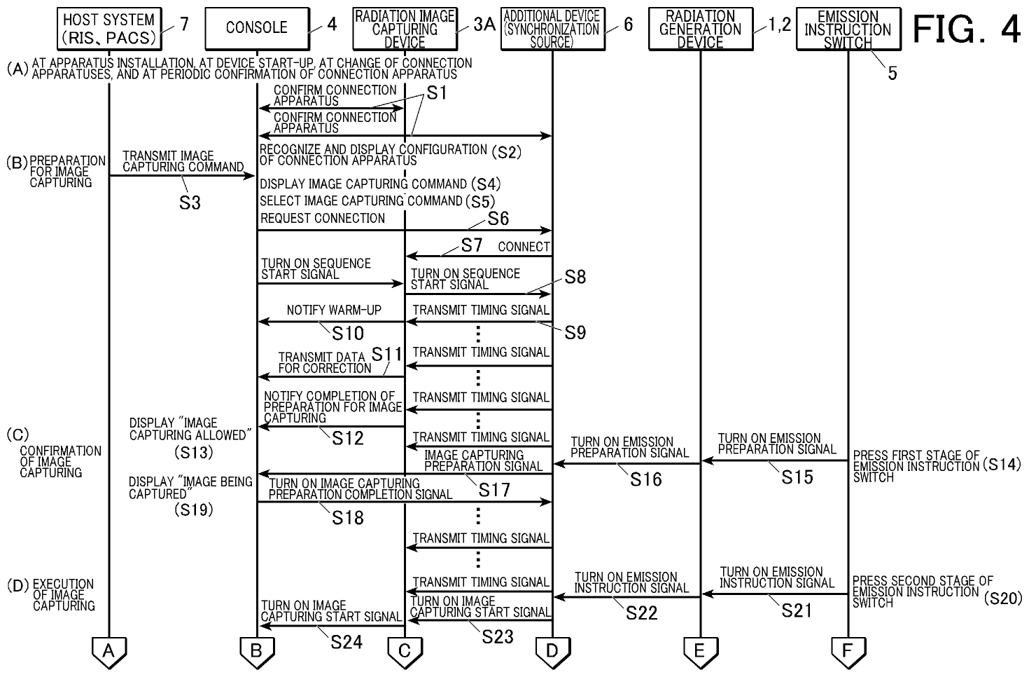

The radiation control console 41 may be communicable with a host system 7 (radiology information system: RIS), a picture archiving and communication system (PACS), and the like (see FIGS. 4, 6, 12, and 14) via an external communication network N such as an in-hospital LAN.

The emission instruction switch 5 is intended for a radiographer to instruct radiation emission.

The emission instruction switch 5 in the present embodiment is manipulated in two stages. Specifically, when the first stage is pressed, the emission preparation signal to be output to the radiation control console 41 is turned on, and when the second stage is pressed, the emission instruction signal to be output to the radiation control console 41 is turned on.

FIG. 1 illustrates the configuration in which the emission instruction switch 5 is connected to the radiation control console 41 so that the emission preparation signal and the emission instruction signal output from the emission instruction switch 5 are input to the radiation controller 11 via the radiation control console 41, whilst the emission instruction switch 5 may be connected to the radiation controller 11 so that the emission preparation signal and the emission instruction signal are directly input to the radiation controller 11.

Operations

Operations of the conventional system 100 will now be described.

Emission Preparation Operation

When the first stage of the emission instruction switch 5 is pressed by the radiographer, the emission instruction switch 5 turns on the emission preparation signal to be output to the radiation controller 11 via the radiation control console 41.

Upon sensing that the emission preparation signal has been turned on, the radiation controller 11 turns on the emission preparation signal to be output to the high voltage generator 12, and brings the emission preparation signal into a state allowed to be output to an external apparatus.

Upon sensing that the emission preparation signal has been turned on, the high voltage generator 12 outputs the emission preparation output to the radiation generator 2.

When the emission preparation output is input, the radiation generator 2 starts preparation for generating radiation.

In a case where a rotary anode is employed as the anode, this preparation for generating radiation indicates an operation of rotating the rotary anode, or the like, for example.

Emission Operation

When the second stage of the emission instruction switch is pressed by the radiographer, the emission instruction switch 5 turns on the emission instruction signal to be output to the radiation controller 11 via the radiation control console 41.

Upon sensing that the emission instruction signal has been turned on, the radiation controller 11 brings this emission instruction signal into a state allowed to be output to an external apparatus, and transmits the emission signal to the high voltage generator 12.

In the case where the radiation controller 11 determines whether to permit radiation emission based on the emission permission signal from the external apparatus, the emission signal is transmitted to the high voltage generator 12 in a case where the emission instruction signal from the emission instruction switch 5 or the radiation control console 41 has been turned on, and the emission permission signal has been received from the external apparatus.

Upon receipt of the emission signal, the high voltage generator 12 applies a high voltage necessary for radiation emission in the radiation generator 2 to the radiation generator 2 (performs an emission output).

When the high voltage is applied from the high voltage generator 12, the radiation generator 2 generates radiation in accordance with the applied voltage.

The generated radiation is adjusted by a controller not shown such as a collimator in terms of the direction of emission, area, radiation quality, and the like, and is emitted to the subject and the cassette 3 behind the subject. Radiation partly passes through the subject to enter the cassette 3.

When radiation enters the cassette 3, a radiation image is formed on a stored film or fluorescent plate.

If the above-described emission preparation signal and the emission instruction signal are turned on at close timings, emission may be performed before rotation of the rotary anode of the radiation generator 2 reaches a sufficient speed, for example, so that a local portion of the rotary anode may be heated excessively to cause a damage to the rotary anode or unstabilize an emitted dose of radiation (to be insufficient or excessive with respect to the emission intensity of electron beams, or the like).

These problems are prevented from occurring by configuring the radiation controller 11 not to transmit the emission signal even upon sensing that the emission instruction signal has been turned on, until a predetermined waiting time elapses upon sensing that the emission preparation signal has been turned on as described above.

In this manner, in radiography through use of the conventional system 100, only a single radiation image (still image) of the subject is captured based on a single image capturing manipulation.

As described above, in the case where the radiation generation device has only one of the pulse emission mode and the continuous emission mode, a device having the pulse emission mode and a device having the continuous emission mode are prepared respectively, and the radiation generation device corresponding to a desired mode is used to capture an image in the desired mode.

In a case where the radiation generation device has both the pulse emission mode and the continuous emission mode, and switches between the modes in the radiation controller 11 or the high voltage generator 12 or switches between the modes by externally making an input to the radiation controller 11 or the high voltage generator 12 or the like, in which mode image capturing is to be performed is selected on the radiation control console 41 when inputting image capturing conditions before image capturing, for example, and the operations of the radiation controller 11 and the high voltage generator 12 are switched before image capturing. Thus, an image is captured in the desired mode.

### First Embodiment

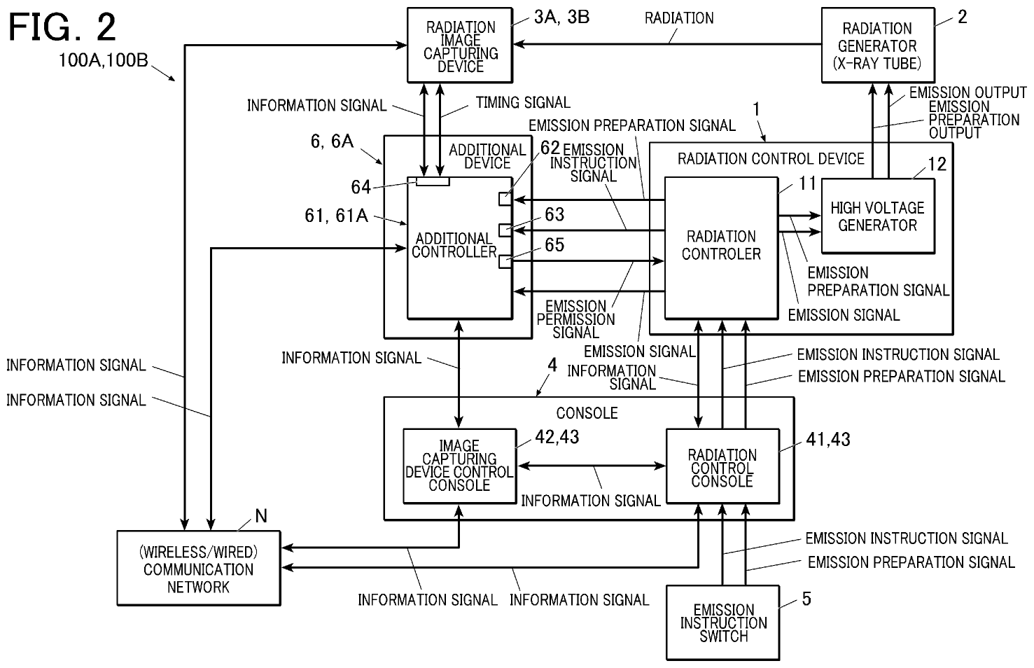

The first embodiment of the present invention will now be described with reference to FIG. 2 to FIG. 6. Components equivalent to those of Conventional technology 1 above will be given identical reference characters, and description thereof will be omitted.

System Configuration

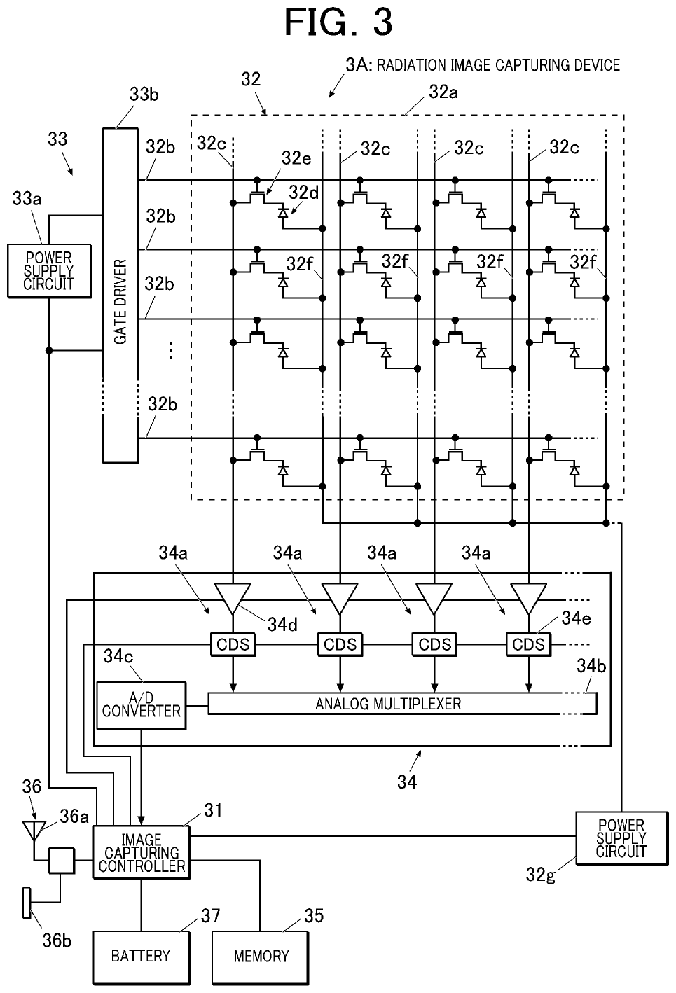

First, a system configuration of a radiography system (hereinafter, a system 100A) according to the present embodiment will be described. FIG. 2 is a block diagram representing the system 100A, and FIG. 3 is a block diagram of an image capturing device 3A. Reference characters in parentheses in FIG. 2 belong to the second embodiment which will be described later.

The system 100A according to the present embodiment includes a radiation image capturing device (hereinafter, an image capturing device 3A) instead of the cassette 3 of the conventional system 100 (see FIG. 1), and further includes an image capturing device control console 42 and an additional device 6, as shown in FIG. 2, for example.

The image capturing device 3A according to the present embodiment includes an image capturing controller 31, a radiation detector 32, a scanning driver 33, a reader 34, a memory 35, a communicator 36, and the like as shown in FIG. 3, in addition to an enclosure and a scintillator, neither shown. The respective components 31 to 36 receive supply of power from a battery 37.

The enclosure is provided with a power switch, a selection switch, an indicator, a connector 36b of the communicator 36 which will be described later, and the like, neither shown.

Upon receipt of radiation, the scintillator emits electromagnetic waves having a wavelength longer than that of radiation, such as visible light.

The image capturing controller 31 includes a computer in which a central processing unit (CPU), a read only memory (ROM), a random access memory (RAM), an input/output interface, and the like, neither shown, are connected to a bus, a field programmable gate array (FPGA), or the like. The image capturing controller 31 may include a dedicated control circuit.

The radiation detector 32 is to generate charges by receiving radiation, and includes a substrate 32a, a plurality of scanning lines 32b, a plurality of signal lines 32c, a plurality of radiation detecting elements 32d, a plurality of switching elements 32e, a plurality of bias lines 32f, a power supply circuit 32g, and the like.

The substrate 32a is formed in a plate shape, and arranged to oppose the scintillator in parallel. The plurality of scanning lines 32b are provided to extend in parallel to each other at a predetermined interval.

The plurality of signal lines 32c are provided to extend in parallel to each other at a predetermined interval, to extend perpendicularly to the scanning lines 32b, and not to be electrically connected to the respective scanning lines.

That is, the plurality of scanning lines 32b and the signal lines 32c are provided to make a lattice.

The radiation detecting elements 32d are to generate electric signals (current, charges), respectively, in accordance with the dose of radiation emitted to the radiation detecting elements (or the amount of light of electromagnetic waves converted in the scintillator), and includes a photodiode, phototransistor, or the like, for example.

The plurality of radiation detecting elements 32d are provided respectively in a plurality of regions sectioned by the plurality of scanning lines 32b and the signal lines 32c on the surface of the substrate 32a. That is, the plurality of radiation detecting elements 32d are arrayed in a matrix form. Thus, the radiation detecting elements 32d are each opposed to the scintillator.

The drain terminal of each of the switching elements 32e which are switching elements is connected to one terminal of each of the radiation detecting elements 32d, and a bias line is connected to the other terminal of each of the radiation detecting elements 32d.

The plurality of switching elements 32e are provided respectively in a plurality of regions sectioned by the plurality of scanning lines 32b and the signal lines 32c, similarly to the radiation detecting elements 32d.

Each of the switching elements 32e has its gate electrode connected to a proximate one of the scanning lines 32b, its source electrode connected to a proximate one of the signal lines 32c, and its drain electrode connected to one terminal of the radiation detecting element 32d in the same region.

The plurality of bias lines 32f are each connected to the other terminal of each of the radiation detecting elements 32d.

The power supply circuit 32g generates a reverse bias voltage, and applies the reverse bias voltage to each of the radiation detecting elements via the bias lines 32f.

The scanning driver 33 includes a power supply circuit 33a, a gate driver 33b, and the like.

The power supply circuit 33a generates an on-voltage and an off-voltage different in voltage for supply to the gate driver 33b.

The gate driver 33b switches a voltage to be applied to each of the scanning lines 32b between the on-voltage and off-voltage.

The reader 34 includes a plurality of readout circuits 34a, an analog multiplexer 34b, an A/D converter 34c, and the like.

The plurality of readout circuits 34a are connected respectively to the respective signal lines 32c of the radiation detector 32, and apply a reference voltage to the respective signal lines 32c.

Each of the readout circuits 34a includes an integration circuit 34d, a correlated double sampling circuit (hereinafter, a CDS circuit) 34e, and the like.

The integration circuit 34d integrates charges discharged to the signal line 32c, and outputs a voltage value in accordance with the amount of integrated charges to the CDS circuit 34e.

The CDS circuit 34e samples and holds the output voltage of the integration circuit 34d before applying the on-voltage to the scanning line 32b to which the radiation detecting element 32d from which a signal is to be read out is connected (while applying the off-voltage), and applies the on-voltage to the relevant scanning line 32b to read out signal charges of the radiation detecting element, and outputs a difference from the output voltage of the integration circuit 34d after applying the off-voltage to the relevant scanning line 32b.

The analog multiplexer 34b outputs a plurality of differential signals output from the CDS circuits 34e to the A/D converter 34c one by one.

The A/D converter 34c sequentially converts image data having an input analog voltage value into image data having a digital value.

The memory 35 includes a static RAM (SRAM), synchronous DRAM (SDRAM), NAND flash memory, hard disk drive (HDD), or the like.

The communicator 36 includes an antenna 36a and the connector 36b for communication with the outside.

The communicator 36 selects which of wireless communication and wired communication is to be performed based on a control signal from the outside. That is, in a case where wireless communication is selected, wireless communication through use of the antenna 36a is performed. In a case where wired communication is selected, information is transmitted/received using a wired LAN or the like. In a case where synchronization is desired to be performed using wired communication, a protocol such as, for example, network time protocol (NTP) or such a method as defined in the international standard IEEE1588 is used to perform synchronization.

When power is turned on, the image capturing device 3A configured in this manner takes any of an “initialized state”, an “accumulation state”, and a “readout and transmission state”. The timing for switching between the states will be described later.

The “initialized state” is a state where the on-voltage is applied to the respective switching elements 32e, and charges generated by the radiation detecting elements 32d are not accumulated in the respective pixels (charges are discharged to the signal lines 32c).

The “accumulation state” is a state where the off-voltage is applied to the respective switching elements 32e, and charges generated by the radiation detecting elements 32d are accumulated in the pixels (charges are not discharged to the signal lines 32c).

The “readout and transmission state” is a state where the on-voltage is applied to the respective switching elements 32e, and the reader 34 is driven to read out image data based on influent charges, and transmit the image data to another device.

The term “transmit” includes both the case of sending image data while holding the image data, and the case of sending image data without holding the image data (what is called forwarding).

Depending on the configuration of elements and devices, accumulated charges are cleared by readout. Thus, “readout” and “initialization” may be performed at the same time as the same operation without differentiating between “readout” and “initialization” as different operations.

Herein, description is given using what is called an indirect image capturing device that converts emitted radiation into electromagnetic waves having another wavelength, such as visible light, to obtain electric signals as an example, whilst the present invention may be what is called a direct image capturing device that converts radiation in the detection elements directly into electric signals.

Other components of the image capturing device 3A are not necessarily be limited to those illustrated in FIG. 3 as long as they are capable of generating image data about a radiation image.

The image capturing device control console 42 transmits/receives an information signal to/from the radiation control console 41 as shown in FIG. 2, and sets subject-related information, image capturing conditions, and the like in the image capturing device 3A.

The radiation control console 41 makes settings in the radiation controller 11, and the image capturing device control console 42 makes settings in the image capturing device 3A, whilst the radiation control console 41 and the image capturing device control console 42 may be collectively referred to as a console 4 in a broad sense in the following description because they make settings for the same image capturing.

The console 4 constitutes the radiation generation control system in the present invention along with the additional device 6.

FIG. 2 illustrates the configuration in which, in the case where settings for image capturing conditions and the like are made on the image capturing device control console 42, the image capturing conditions and the like are set in the radiation controller 11 via the radiation control console 41 (the radiation control console 41 and the image capturing device control console 42 transmit/receive an information signal to/from each other), whilst settings in the radiation controller 11 may be directly made from the image capturing device control console 42.

Settings in the image capturing device 3A may be made from the radiation control console 41.

FIG. 2 illustrates the configuration in which the console 4 is connected to the image capturing device 3A via the additional device 6, whilst the console 4 may be connected directly to the image capturing device 3A, or may be connected to the image capturing device 3A via the communication network N, as shown in FIG. 2, for example.

The console 4 is capable of setting the operation of the additional device 6.

Specifically, the number of times of output at which a timing signal is output (the maximum number of captured images N) before turning on the emission permission signal (a third signal in the present invention) output from the additional device 6 to the radiation generation device, or an output time between turn-on and turn-off of the output of the emission permission signal is set in the additional device 6.

The console 4 may be provided with a display 43 to cause the display 43 to display the number of times of output or output time set in the additional device 6.

The console 4 may cause the display 43 to display the fact that emission is allowed when an image capturing start signal (a second signal in the present invention, details of which will be described later) input to the additional device 6 is turned on.

The console 4 may cause the display 43 to display the fact that radiation is being emitted while the additional device 6 is outputting the emission permission signal.

The additional device 6 is the radiation generation control device in the present invention, and includes an additional controller 61 having a first acquisitor 62, a second acquisitor 63, a first connector 64, and a second connector 65.

The additional controller 61 exerts integrated control over the operation of each component of the additional device 6 by means of a CPU, RAM, and the like.

In this case, various processing programs held in a memory not shown are read out for expansion into the RAM, and various types of processing are executed in accordance with the processing programs.

The first acquisitor 62 makes a contact (for example, a connector) with the radiation controller 11, and in the present embodiment, acquires the emission preparation signal output from the emission instruction switch 5 via the radiation controller 11 (radiation generation device).

The second acquisitor 63 makes a contact (for example, a connector) with the radiation controller 11, and in the present embodiment, acquires the emission instruction signal output from the emission instruction switch 5 via the radiation controller 11 (radiation generation device).

As described above, since the emission instruction signal corresponds to the first signal in the present invention, the second acquisitor 63 constitutes an acquisitor in the present invention.

The first connector 64 makes a contact (for example, a connector) with the image capturing device 3A, and inputs an emission start signal.

The emission start signal is a signal turned on when the image capturing device 3A is brought into a state allowed to perform image capturing, and is turned off when the image capturing device 3A is brought into a state not allowed to perform image capturing, which is a signal indicating a driving state of the image capturing device 3A in the present invention.

The first connector 64 makes a contact (for example, a connector) with the image capturing device 3A, and transmits/receives an information signal to/from the image capturing device 3A. As the information signal, information concerning selection of an image capturing operation mode of the image capturing device 3A or information concerning image capturing conditions such as an image capturing frame rate, for example, are transmitted/received.

The second connector 65 is a connector in the present embodiment, which is connectable to the radiation controller 11 (radiation generation device) by inserting therein one end of a cable having the other end connected to the radiation controller 11 (radiation generation device).

The second connector 65 outputs the emission permission signal to the radiation controller 11.

FIG. 2 illustrates the configuration in which the first acquisitor 62, the second acquisitor 63, the first connector 64, and the second connector 65 directly transmit/receive information and signals to/from other devices (the first and second acquisitors 62, 63 and the second connector 65 to/from the radiation control device 1, and the first connector 64 to/from the image capturing device 3A), whilst at least any of the first acquisitor 62, the second acquisitor 63, the first connector 64, and the second connector 65 may be connectable to another device via a relay not shown that relays a signal.

The relay may be a wired/wireless communication network, for example. This may be the communication network N shown in FIG. 2, or connection may be made via another communication network not shown.

FIG. 2 also illustrates the case in which the first acquisitor 62, the second acquisitor 63, the first connector 64, and the second connector 65 are provided separately, whilst at least two of the first acquisitor 62, the second acquisitor 63, the first connector 64, and the second connector 65 may be formed integrally (the respective components 62 to 65 may be shared).

The additional controller 61 of the additional device 6 configured in this manner maintains the emission permission signal that instructs emission of radiation to be output from the second connector 65 to the radiation controller 11 in the on state for a predetermined period based on the emission instruction signal acquired from the radiation controller 11 via the second acquisitor 63 and the emission start signal input from the image capturing device 3A via the first connector 64.

The additional controller 61 may be prevented from outputting the emission permission signal even upon sensing that the image capturing start signal has been turned on, until a predetermined waiting time elapses upon sensing that the emission start signal has been turned on.

The additional controller 61 causes a timing signal (a fourth signal in the present invention) that instructs a timing of capturing a radiation image to be output from the first connector 64 to the image capturing device 3A while the emission permission signal is maintained in the on state.

The image capturing timing is a timing of starting an operation of accumulating charges of a radiation image, for example. That is, the image capturing device 3A according to the present embodiment starts accumulation of charges in accordance with the timing signal, and sequentially performs operations of terminating accumulation, reading out charges in each pixel, imaging charges in each pixel, and storing and transmitting images by means of a timer of the image capturing device 3A.

Such control enables the additional controller 61 to control an accumulation timing of accumulating charges at the time of radiation emission by means of the timing signal. As a result, for a period in which radiation is being emitted, charges produced by radiation emission are accumulated reliably, and eventually, an image produced by radiation emission is acquired reliably.

In the case where the start of the charge accumulating operation is the above-described image capturing timing in this manner, the image capturing device 3A may wait in a state allowed to transition to the accumulation timing corresponding to the image capturing operation by means of radiation emission, and may start the accumulating operation in accordance with the timing signal. For example, the image capturing device 3A repeats a reset operation of applying on-charges to the switching elements 32e in order to discharge dark charges which are noise components accumulated over time in the respective pixels prior to accumulation of charges by means of radiation emission to the outside of the pixels. Thus, by setting the charge accumulating timing in this manner, the image capturing device 3A terminates the reset operation at the above-described image capturing timing, and transitions to the operation of successively performing accumulation of charges in accordance with radiation and imaging through readout.

Such control enables the additional controller 61 to reliably acquire an image produced by radiation emission, similarly to the above-described case.

The image capturing timing triggered by input of the timing signal may be a timing when starting any of various operations repeatedly performed by the image capturing device 3A besides the above-described charge accumulating operation.

For example, in a case where charges accumulated in each pixel need to be reset before the accumulating operation, a timing of starting reset may be the above-described image capturing timing

In this case, the image capturing device 3A may sequentially transition to the accumulating operation after reset is completed.

Such control enables the accumulating operation of accumulating charges produced by radiation emission to be started in a state where dark charges which are noise components accumulated over time in the respective pixels prior to accumulation of charges produced by radiation emission have been discharged by reset, so that a less noisy image is acquired.

Alternatively, a timing for terminating the accumulating operation may be the above-described image capturing timing Alternatively, a timing for starting readout of accumulated charges in response to the timing signal may be the above-described image capturing timing.

Such control enables the additional controller 61 to control both the timing for radiation emission in response to the emission permission signal and the image capturing operation (terminating the reset operation, starting the accumulating operation, terminating the accumulating operation, starting readout of accumulated charges, and the like) in response to the timing signal. As a result, charges produced by radiation emission are accumulated reliably, and eventually, an image produced by radiation emission is acquired reliably.

The timing signal may be used for terminating each operation, rather than for starting. For example, the accumulating operation may be started at a timing when the timing signal changes from OFF to ON, and the accumulating operation may be terminated at a timing when the timing signal changes from ON to OFF.

Such control enables the additional controller 61 to reliably acquire an image produced by radiation emission, similarly to each of the above-described cases.

In the present embodiment, the timing signal is repeatedly output.

In the present embodiment, the additional controller 61 determines the length of the predetermined period in accordance with an image capturing time or the number of captured images previously set. That is, the emission permission signal is maintained in the on state until the number of times of outputting the timing signal reaches a predetermined number of times of output, or until a predetermined output time elapses after the timing signal is output first.

The additional controller 61 may have a timer for controlling timing for repeatedly transmitting the timing signal in a predetermined cycle.

The additional controller 61 may have a counter that counts the number of outputs for repeatedly outputting the timing signal until a predetermined number of times of output is reached. Alternatively, the additional controller 61 may have a timer for repeatedly outputting the timing signal until a predetermined output time elapses after the timing signal and the emission permission signal are output first.

The timing signal may be output in a phase before the second stage of the emission instruction switch 5 is pressed (the emission instruction signal is acquired).

Specifically, for example, the timing signal may also be output after a sequence start signal (a fifth signal in the present invention) is acquired (upon sensing that the sequence start signal has been turned on) and before the emission instruction signal is acquired, or after the emission preparation signal (a sixth signal in the present invention) is acquired (upon sensing that the emission preparation signal has been turned on) and before the emission instruction signal is acquired.

Operations

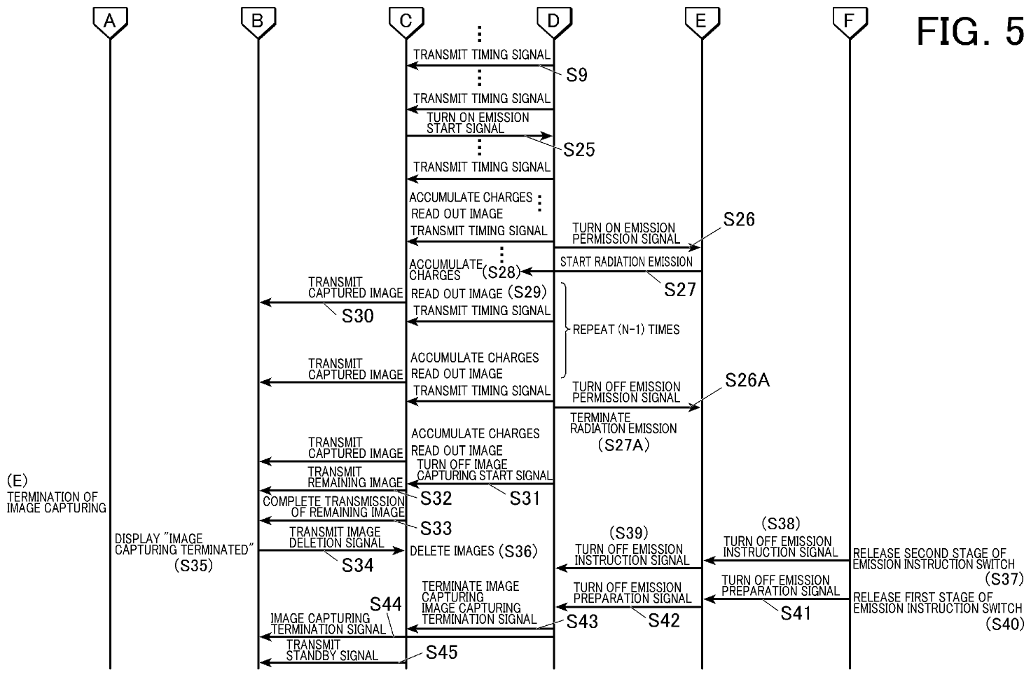

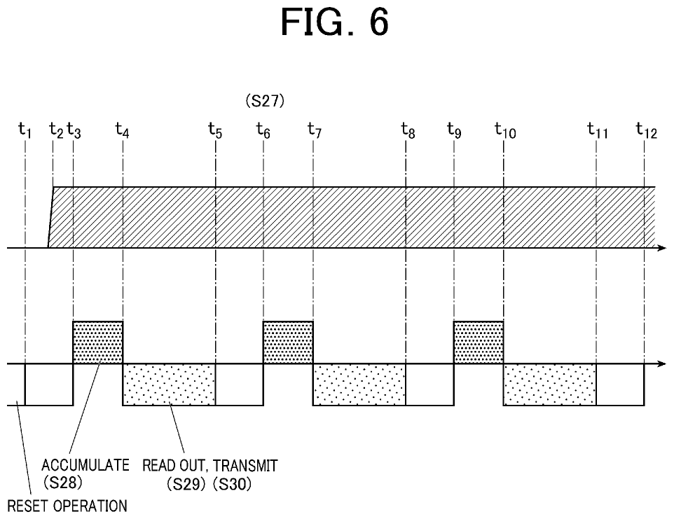

Operations of the above-described system 100A will now be described. FIG. 4 and FIG. 5 are ladder charts representing the operations of the system 100A according to the present embodiment, and FIG. 6 is a timing chart representing the operations of the system 100A.

A) At Apparatus Installation, at Device Start-Up, at Change of Connection Apparatuses, and at Periodic Confirmation of Connection Apparatus

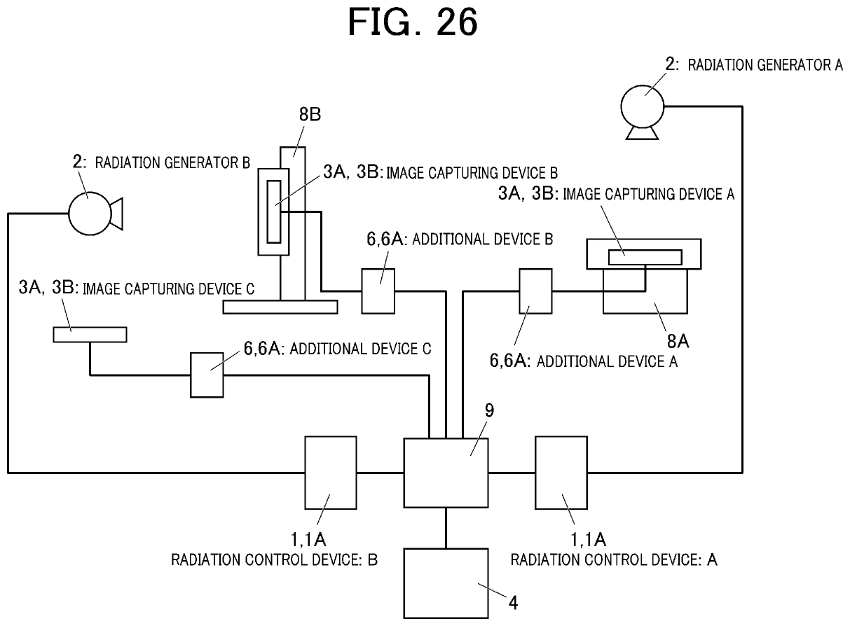

First, the console 4, in particular, the image capturing device control console 42 confirms the image capturing device 3A and the additional device 6 connected to an image capturing environment being controlled by the console 4 at apparatus installation, at start-up of the image capturing system, at change of connection apparatuses, and besides, at periodic confirmation of a connection apparatus (step S1) as shown in FIG. 4, and causes the display 43 of the console 4 to display an apparatus configuration and a connection configuration (step S2). As the apparatus configuration and connection configuration, those shown in FIG. 26, for example, are displayed on the display 43 of the console 4.

Confirmation of the image capturing device 3A and the additional device 6 connected to the image capturing environment being controlled by the console 4 is performed by making a request from the console 4 to the image capturing device 3A and the additional device 6 for the presence/absence of connection and ID, and the image capturing device 3A and the additional device 6 returning the presence/absence of connection and the ID, for example.

As the ID, an apparatus-specific ID such as a MAC address set in an apparatus-specific manner, an apparatus-specific BSSID, or a serial number set in a device-specific manner can be used, or an ID set afterward such as a set IP address or a set ESSID can be used, for example.

B) Preparation for Image Capturing

Thereafter, upon receipt of an image capturing command from the host system 7 such as RIS or HIS (step S3), the console 4 causes the received image capturing command to be displayed on the screen of the console 4 (step S4).

On that occasion, an operator may be notified that a new image capturing command has been received using light or sound.

The radiographer performs a manipulation such as changing an image capturing order based on the displayed image capturing command, and selects an image capturing command for which image capturing is performed next (step S5).

On that occasion, the image capturing device 3A to be used may be selected from among the plurality of image capturing devices 3A being connected.

The image capturing device 3A recommended in accordance with an image capturing technique of the radiographer may be selected automatically from among the plurality of image capturing devices 3A being connected.

In a case where there is no particular change, the image capturing device 3A used in previous image capturing may be selected continually.

When the image capturing device 3A to be used is selected, the console 4 makes a connection request to each of the image capturing device 3A and the additional device 6 (step S6).

Upon receipt of the connection request, the image capturing device 3A and the additional device 6 each connect to the console 4 (step S7).

The connection request may be made from the console 4 to the additional device 6, and may be further made from the additional device 6 to the image capturing device 3A, as shown in FIG. 2.

The image capturing device 3A and the console 4 are connectable via the communication network N as shown in FIG. 2 or directly connectable. However, if the console 4 and the image capturing device 3A are connected directly, the console 4 may be connected to the image capturing device 3A not connected to the additional device 6, and a connection configuration in a state where the additional device 6 and the image capturing device 3A cooperate with each other may not be established.

By connecting the image capturing device 3A and the console 4 via the additional device 6 in the above manner, the console 4 is reliably connected to the image capturing device 3A connected to the additional device 6.

Alternatively, although illustration is omitted, the connection request may be made from the console 4 to each of the image capturing devices 3A, and thereafter, the connection request may be made from each of the image capturing devices 3A to the additional device 6.

Since the image capturing device 3A to be used for image capturing is set on the console 4, the above configuration allows the image capturing device 3A to be used to be reliably selected and connected to the additional device 6, and a state where the additional device 6 and the image capturing device 3A to be used cooperate with each other is established without selecting an incorrect image capturing device 3A.

With the above configuration, the image capturing device 3A is selected not only from among the image capturing devices 3A connected to the additional device 6 as described above, but also from among all of the available image capturing devices 3A, and connection is made.

When starting connection, the image capturing device 3A may automatically have its own state transition from the aforementioned low-power consumption mode in which preparation for image capturing or image capturing is allowed to a mode in which higher power than in the low-power consumption mode is consumed.

Subsequently, the radiographer causes image capturing conditions necessary for image capturing to be transmitted from the console 4 to at least one of the image capturing device 3A, the additional device 6, and the radiation control device 1.

The image capturing conditions include, for example, information concerning the operation mode as to which operation the image capturing device 3A performs based on the timing signal, information concerning the capturing frame rate and the like, information concerning emission of radiation such as the voltage, current, emission time, and the like at image capturing, information concerning the operation mode as to whether the radiation emission device performs emission in the pulse emission mode or in the continuous emission mode, and the like.

The image capturing conditions are transmitted using an information signal exchanged between the console 4 and the additional device 6 and an information signal exchanged between the additional device 6 and the image capturing device 3A, for example.

Upon receipt of the image capturing conditions, at least one of the image capturing device 3A, the additional device 6, and the radiation control device 1 sets the image capturing conditions.

The console 4 may have a function of storing combinations of these pieces of information with which the operations can be performed or a conditional range, and performing selection in accordance with these combinations or the conditional range, or confirmation through collation with the combinations or the conditional range, or selection permission control in accordance with these combinations or the conditional range.

Subsequently, when the radiographer instructs the console 4 to start image capturing, the console 4 turns on the sequence start signal that instructs the image capturing device 3A and the additional device 6 to start the image capturing sequence, and transmits the sequence start signal to the image capturing device 3A and the additional device 6 (step S8). The sequence start signal is transmitted using the information signal exchanged between the console 4 and the additional device 6 and the information signal exchanged between the additional device 6 and the image capturing device 3A, for example.

Upon sensing that the sequence start signal has been turned on, the image capturing device 3A and the additional device 6 start preparation for image capturing.

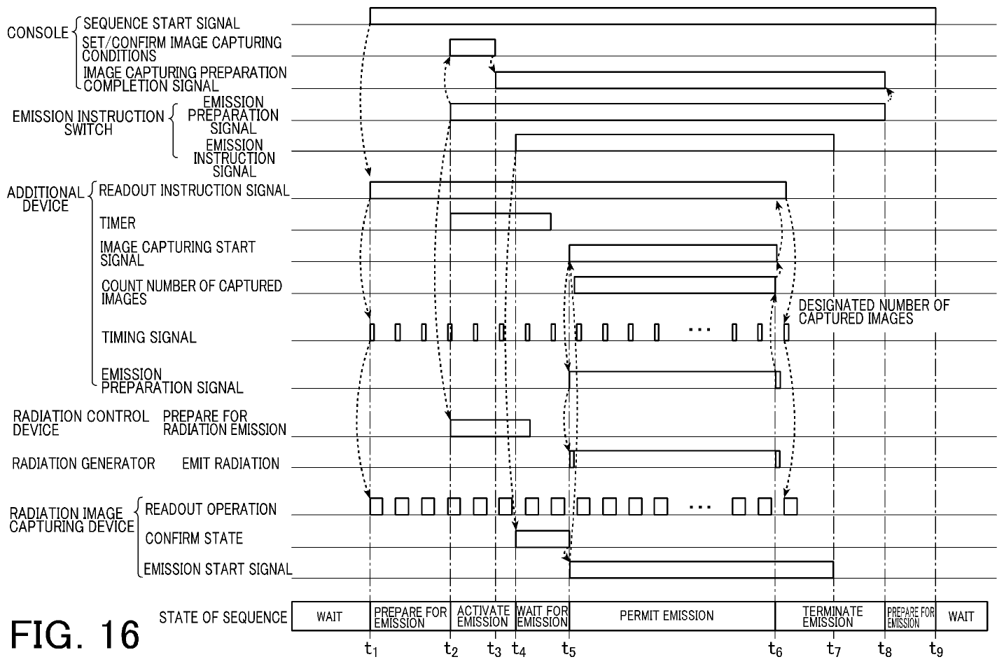

In a case where the additional device 6 transmits the timing signal in a phase before the emission instruction switch 5 is pressed, control may be exerted such that the additional device 6 turns on a readout instruction signal (see FIG. 16) upon sensing that the sequence start signal has been turned on, and transmits the timing signal repeatedly to the image capturing device 3A at a predetermined interval (step S9).

Each time this timing signal is received, the image capturing device 3A repeats the readout operation. Then, the circuit in the image capturing device 3A rises in temperature. That is, the readout operation repeatedly performed by the image capturing device 3A in this phase warms up the image capturing device 3A.

In an initial phase in which the image capturing device 3A repeats the readout operation, the console 4 is notified that warm-up has been started (step S10).

A readout operation (reset operation) for removing charges accumulated immediately before image capturing needs to be performed for the image capturing device 3A.

Since the image capturing device 3A consumes power when performing the readout operation, the image capturing device 3A rises in temperature accordingly. With this temperature rise, particularly a light receiver of the image capturing device 3A is changed in sensitivity, and the image density to be output to the amount of entering radiation is also changed. If a single still image is captured, image changes due to this temperature rise will not come into question, but in the case of performing kymography (repeated capturing of still images) as in the system 100A according to the present embodiment, image changes due to the temperature rise during image capturing come into question.

However, in the case of performing warm-up as described above, changes in image density due to such a temperature rise are reduced.

At any timing while step S9 is repeated multiple times, the image capturing device 3A may acquire an image for correction.

For example, in the case where the image capturing device 3A performs warm-up (performs the readout operation before pressing the emission instruction switch 5), an image read out in the latter half of this warm-up may be transmitted to the console 4 as an image for correction (step S11).

A plurality of pixels included in the image capturing device 3A have different properties from each other, and even in a state where radiation is not emitted, the level of charges equivalent to the image brightness is different between pixels. Therefore, by acquiring the image read out in the latter half of warm-up as an image for correction, and subtracting each signal value of the image for correction from each signal value of a captured image obtained later, for example, a captured image from which variations between pixels have been removed is obtained.

Herein, the case of simply subtracting an image for correction from a captured image has been described as an example as the method of using the image for correction, whilst noise components can be removed using various computations.

When in the readout operation (reset operation) for removing charges accumulated immediately before image capturing except acquisition of the image for correction, at least one of the step of imaging the removed charges, the step of storing the imaged image data in the memory in the image capturing device 3A, and the step of transferring the imaged data or image data stored in the memory in the image capturing device 3A to the console 4 may be performed as an operation similar to normal image capturing.

Accordingly, there are few differences when performing image capturing in subsequent steps because performing the above-described steps similar to those of normal image capturing achieves conditions closer to actual image capturing, and an influence caused by the temperature rise, for example, is reduced.

On the other hand, in the case of performing the above-described steps similar to those of normal image capturing, problems arise in that more power is consumed, the capacity of the memory allowed to be used during image capturing is reduced because images when useless radiation is not emitted are stored in the memory of the image capturing device 3A, images when useless radiation is not emitted are transmitted to the console to occupy part of the communication capacity, and the capacity of the memory allowed to be used during image capturing is reduced because the images are stored in the memory of the console. In order to avoid occurrence of such problems, part of the above-described steps may be omitted.

Particularly in recent years, the memory of the image capturing device 3A increases in capacity, and the image capturing controller 31 is improved in computational capability. Therefore, concerning the aforementioned image correction, at least part of steps may be performed in the image capturing device 3A after image capturing, and an image having undergone correction processing may be sent to the console 4. In that case, it is not necessary to send the aforementioned image for correction to the console, but may be stored in the memory of the image capturing device 3A.

The image capturing device 3A completes warm-up when the number of times of readout previously set as warm-up is reached or a readout operation period elapses. By avoiding turning on the emission start signal in step S25 (see FIG. 5) of turning on the emission start signal which will be described later, for example, until the aforementioned number of times of readout previously set is reached or the readout operation period elapses, image capturing through emission of radiation is prevented from being started until warm-up is completed.

Thereafter, the image capturing device 3A notifies the console 4 that preparation for image capturing has been completed (step S12).

On that occasion, “image capturing allowed” may be displayed on the display 43 of the console 4 (step S13).

C) Confirmation of Image Capturing

The additional device 6 continuously transmits the timing signal repeatedly to the image capturing device 3A, and the image capturing device 3A repeats the readout operation of the image capturing device 3A each time this timing signal is received.

When the radiographer finishes positioning of the subject, and presses the first stage of the emission instruction switch 5 (step S14), the emission instruction switch 5 turns on the emission preparation signal to be output to the radiation controller 11 via the console 4 (step S15).

Upon sensing that this emission preparation signal has been turned on, the radiation controller 11 of the radiation generation device turns on the emission preparation signal to be output to the high voltage generator 12 and the additional device 6 (step S16). Accordingly, the first acquisitor 62 of the additional device 6 acquires the emission preparation signal (output before the emission instruction signal and after the sequence start signal is turned on).

In this manner, the radiation generation device including the radiation controller 11 starts preparation for radiation emission in response to the emission preparation signal.

Upon sensing that the emission preparation signal from the radiation controller 11 has been turned on, the additional controller 61 of the additional device 6 transmits an image capturing preparation signal to the console 4 (step S17).

Upon receipt of the image capturing preparation signal, the console 4 starts preparation for image capturing. Preparation for image capturing in the console 4 is an operation of confirming that settings in the image capturing device control console 42 that constitutes the console 4, for example, and the radiation control console 41 that controls radiation emission are equal, or confirming that designated image capturing conditions or the like have been set in the image capturing device 3A.

When preparation for image capturing is terminated, the console 4 turns on an image capturing preparation completion signal to be output to the additional device 6 (step S18).

On that occasion, “image being captured” may be displayed on the display 43 of the console 4 (step S19).

In this phase where preparation for image capturing is completed, input to the console 4 for changing the image capturing conditions or the like may be locked so as to prevent a change from being made.

In the case of still image capturing, image capturing is terminated in a short time, and thus, the risk that a change in image capturing conditions or the like is made during image capturing is small, and the necessity to configure in this manner is low. In the case of kymography, however, the image capturing period is long, which increases the risk that the radiographer or a third party other than the radiographer manipulates the console screen with or without intention to change the image capturing conditions or the like.

Therefore, by locking input to the console 4 for changing the image capturing conditions or the like from this phase to the termination of the sequence after step S45 which will be described later, such a change in image capturing conditions is reliably prevented.

Although FIG. 4 illustrates the case in which the image capturing preparation signal is output from the additional device 6 to the console 4, there is a case in which the image capturing preparation signal is output to the image capturing device 3A rather than the console 4 to cause the image capturing device 3A to perform preparation for image capturing, and when preparation for image capturing in the image capturing device 3A is completed, the image capturing preparation completion signal is output from the image capturing device 3A to the additional device 6.

There is another case in which the image capturing preparation signal is output to each of the console 4 and the image capturing device 3A to cause them to perform preparation for image capturing, and when preparation for image capturing in both of them is completed, the image capturing preparation completion signal is transmitted from each of the console 4 and the image capturing device 3A to the additional device 6, and in a phase where the additional device 6 receives the image capturing preparation completion signals from both of them, it is determined that the whole preparation for image capturing has been completed.

Although illustration is omitted, in a case where the radiation controller 11 of the radiation generation device has a connector that inputs the image capturing preparation completion signal with which it is input that preparation for image capturing in an external apparatus has been completed, the additional device 6 may output the image capturing preparation completion signal to this connector of the radiation controller 11.

When the radiation controller 11 senses that the image capturing preparation completion signal from the additional device 6 has been turned on, it is sensed that the image capturing device 3A is in a state allowed to perform image capturing. By exerting control such that the radiation control device 1 performs radiation emission upon sensing that the image capturing preparation completion signal has been turned on, the danger that radiation is emitted in a state where the image capturing device 3A is not allowed to perform image capturing to expose the subject to radiation uselessly is reliably eliminated.

D) Execution of Image Capturing

Subsequently, when the radiographer presses the second stage of the emission instruction switch 5 (step S20), the emission instruction switch 5 turns on the emission instruction signal to be transmitted to the radiation controller 11 via the console 4 (step S21).

At this time, the additional device 6 continuously transmits the timing signal repeatedly to the image capturing device 3A, and the image capturing device 3A repeats the readout operation each time this timing signal is received.

Since the emission permission signal from the additional device 6 is in the off state at this point of time even if the emission instruction signal is input from the emission instruction switch 5, the radiation controller 11 of the radiation generation device does not transmit the emission signal to the high voltage generator 12.

The radiation controller 11 turns on the emission instruction signal to be transmitted to the additional controller 61 (step S22).

Upon receipt of the emission instruction signal, the additional device 6 turns on the image capturing start signal to be output to the image capturing device 3A and the console 4 for notifying whether to permit start of image capturing (steps S23, S24).

Upon sensing that the image capturing start signal has been turned on, the image capturing device 3A is triggered by the termination of the readout operation being performed by itself at that point of time to turn on the emission start signal to be output to the additional device 6, as shown in FIG. 5, for example (step S25). This is because the readout operation of the image capturing device 3A is to sequentially read out charges accumulated in pixels arranged two-dimensionally to acquire an image of the whole light receiving surface, and if the emission start signal is turned on in the middle of readout so that radiation is emitted, a difference occurs in signal value between pixels in which readout has been completed and pixels in which readout has not been completed, which results in significant degradation in image quality.

In the present embodiment, radiation emission and image readout of the image capturing device 3A are performed based on the emission permission signal and the timing signal from the additional device 6 as will be described later. Thus, radiation emission in the middle of the readout operation does not occur in a normal routine. Thus, the emission start signal may be turned on without considering the readout timing of the image capturing device 3A described above.

The image capturing device 3A repeats the image readout operation even after the emission start signal is turned on. An image read out after the emission start signal is turned on is stored in the memory of the image capturing device 3A or transmitted to the console 4 as a captured image.

Upon sensing that the emission start signal from the image capturing device 3A has been turned on, the additional device 6 determines that the image capturing device 3A is in a state allowed to perform image capturing, and turns on the emission permission signal being output to the radiation controller 11 (step S26).

When the emission permission signal is turned on, the image capturing instruction signal and the emission permission signal are completed. Thus, the radiation controller 11 of the radiation generation device turns on the emission signal being output to the high voltage generator 12.

When the emission signal is turned on, the high voltage generator 12 continuously generates a high voltage necessary for radiation emission, and continues outputting the high voltage to the radiation generator 2 as an emission output.

When the emission output is input, the radiation generator 2 continues emitting radiation to the image capturing device 3A (step S27).

The emitted radiation passes through the subject not shown located between the image capturing device 3A and the radiation generator 2, and enters the image capturing device 3A.

Each time the timing signal is received, the image capturing device 3A accumulates an amount of charges in accordance with the intensity of entered radiation (step S28), and reads out the charges as a captured image (step S29).

Thereafter, step S28 and step S29 are repeated N-1 times (repeated N times (the maximum number of captured images N) in total).

The image capturing device 3A transmits the radiation image having been read out to the console 4 (step S30).

In the case of transmitting the captured image to the console 4, and in a case where transmission to the console 4 is not on time because of the data amount or a communication environment, some captured images among a plurality of captured images or part of a single captured image may be stored in the image capturing device 3A, and the rest may be transmitted to the console 4 in this step S30.

In this step S30, a captured image may be stored in the memory of the image capturing device 3A without transmitting the captured image during image capturing. The stored captured image may be transmitted to the console 4 via a wired connection, a wireless connection, or a portable memory removably attached to the image capturing device 3A after image capturing.

An example of the operation of the image capturing device 3A will now be further described using FIG. 6. Herein, the aforementioned case of starting accumulation of charges in accordance with the timing signal will be described.

### Reset Operation/Correction Image Acquring Operation

In the reset operation performed before image capturing, the image capturing device 3A repeatedly performs the aforementioned operation in a state where there is no radiation emission from the radiation generation device to discharge charges (dark charges or dark current) accumulated in each pixel and not based on radiation emission. Accordingly, the charges accumulated in each pixel and not based on radiation emission which are noise components for an image generated by charges based on radiation emission are reset. When in this reset operation, control may be exerted such that charges entered in the reader 34 are not converted into image data (the operation prior to t1).

In a correction image acquiring operation performed before or after image capturing, the image capturing device 3A repeatedly performs the aforementioned operation in a state where there is no radiation emission from the radiation generation device to discharge charges (dark charges or dark current) accumulated in each pixel and not based on radiation emission. Accordingly, the charges accumulated in each pixel and not based on radiation emission which are noise components for an image generated by charges based on radiation emission are reset. When in this correction image acquiring operation, by converting the entered charges in the reader 34 into image data and storing the image data, the amount of noise components in the state where there is no radiation emission is stored, and by subtracting these noise components from image data in a state where radiation has been emitted, the noise components are removed. This correction image acquiring operation may be performed as part of the aforementioned reset operation (the operation prior to t1).

Operation of Terminating Reset Operation/Correction Image Acquring Operation

When the emission instruction signal in step S21 is turned on subsequently to the reset operation or correction image acquiring operation, the image capturing start signal to be input to the image capturing device 3A is turned on (step S23). Then, the image capturing device 3A stops the aforementioned reset operation or correction image acquiring operation.

In a case where the correction image acquiring operation has not completed acquisition of a predetermined number of images for correction even if the image capturing start signal has been turned on, the correction image acquiring operation is not stopped, but the correction image acquiring operation is continued until the predetermined number of images for correction are acquired, and thereafter, the correction image acquiring operation is stopped.

Alternatively, the reset operation or correction image acquiring operation is sequentially performed for the radiation detecting elements 32d arranged two-dimensionally. Therefore, the aforementioned reset operation or correction image acquiring operation may be stopped in a phase where the reset operation or correction image acquiring operation for the radiation detecting element 32d at the end of the radiation detecting elements 32d arranged two-dimensionally is terminated. This is because, if the reset operation or correction image acquiring operation is stopped at a position other than the end of the radiation detecting elements 32d arranged two-dimensionally, a time elapsed after the reset operation or correction image acquiring operation is performed and before image capturing is performed is greatly different between adjacent radiation detecting elements 32d among the radiation detecting elements 32d arranged two-dimensionally, and thus, a step difference may occur in an acquired image in this portion. By stopping the reset operation or correction image acquiring operation in the phase where the reset operation or correction image acquiring operation is terminated for the radiation detecting element 32d at the end of the radiation detecting elements 32d arranged two-dimensionally, such a step difference is prevented from occurring.

When the reset operation or correction image acquiring operation is stopped, the image capturing device 3A turns on the emission start signal in step S25 to notify that the reset operation or correction image acquiring operation has been stopped, and the state allowed to perform image capturing has been brought about (t1).

Image Capturing Operation

Thereafter, the additional controller 61 turns on the emission permission signal being output to the radiation generation device (step S26).

Then, the radiation generation device starts emission of radiation to the image capturing device 3A (step S27; t2).

The image capturing device 3A generates charges upon receipt of radiation. However, since the on-voltage is being applied to the switching elements 32e in this phase, the image capturing device 3A discharges the generated charges as they are to the reader 34.

Upon receiving the timing signal from the additional controller 61, the image capturing device 3A applies the off-voltage to each of the scanning lines 32b to transition to a state where the charges generated by the radiation detecting elements 32d are accumulated in pixels (t3, t6, t9, . . . ) as shown in FIG. 6, and accumulates the generated charges in each pixel (step S28).

The image capturing device 3A continues the mode of accumulating charges for a predetermined time by means of its own timer (t3 to t4, t6 to t7, t9 to t10 . . . ).

After the predetermined time elapses upon application of the off-voltage, the image capturing device 3A applies the on-voltage to each of the switching elements 32e after the above-described predetermined time elapses to perform the readout operation of discharging charges accumulated in each pixel to the signal lines 32c. In the readout operation, the image capturing device 3A reads out image data based on entered charges by the reader 34 for conversion into image data. At least part of image data converted into image data may be transmitted to the console 4, or may be stored in the memory 35 of the image capturing device 3A (steps S29, S30; t4 to t5, t7 to t8, t10 to t11, . . . ).

Another Embodiment of Operation Control System

Although the above description shows an example in which the image capturing device 3A transitions to the state where charges are accumulated in pixels in response to the timing signal from the additional controller 61 (the example of performing the operations at t3, t6, t9, . . . in response to the timing signal), control may be exerted to perform another operation in response to this timing signal. That is, a transition may be made such that the image capturing device starts reading out accumulated charges in response to the timing signal (performs the operations at t4, t7, t10 . . . in response to the timing signal).

For example, control may be exerted to cause the image capturing device 3A to transition to the state where charges are accumulated in pixels (t6 to t7, t9 to t10, . . . ) after the readout operation of discharging charges accumulated in each pixel to the signal lines 32c (t4 to 5, t7 to t8, t10 to t11, . . . ) is terminated, without waiting for the timing signal from the additional controller 61.

Then, after the additional controller 61 turns on the emission permission signal being output to the radiation generation device, control is exerted to output the timing signal.

Then, a transition is made to the readout operation of discharging charges accumulated in each pixel to the signal lines 32c in response to the timing signal the image capturing device 3A has received (t4, t7, t10, . . . ).

Also by repeating such operation control, image capturing is performed while synchronizing the radiation emission timing of the radiation generation device and the image generation timing of the image capturing device.

E) Termination of Image Capturing

The additional device 6 counts the number of times of transmitting the timing signal after a point of time when the emission permission signal is turned on (emission of radiation is started) or a time after a point of time when the emission permission signal is turned on, and in each case, it is determined whether the maximum number of captured images N or the maximum image capturing time previously set has been reached. In a case where it is determined that the counted number of times of transmitting the timing signal (the number of already captured images) has reached the maximum number of captured images N, or the counted time has reached the maximum image capturing time, the emission permission signal being output to the radiation generation device is turned off (step S26A). Then, the radiation generation device terminates emission of radiation (step S27A).

The additional device 6 turns off the image capturing start signal (step S31) to stop output of the aforementioned timing signal.

After the additional device 6 outputs the last timing signal, control may be exerted to perform an operation at least once in which the image capturing device 3A accumulates charges in an amount in accordance with the intensity of entered radiation (step S28), and the charges are read out as a captured image (step S29). Accordingly, an image to which radiation is emitted last is reliably read out to obtain a captured image, and the subject is reliably prevented from being exposed to radiation uselessly.

After the additional device 6 outputs the last timing signal, control may be exerted to perform an operation in which the image capturing device 3A accumulates charges (step S28), and the charges are read out as a captured image (step S29), and then, the image capturing device 3A further accumulates charges, and the charges are read out as a captured image. These captured images are images captured in a state where radiation is not emitted, and thus, they are used for correcting a captured image when radiation is emitted similarly to the aforementioned image for correction.

That is, an image captured before image capturing through use of radiation may be used as an image for correction as described above, or an image captured after image capturing through use of radiation may be used as an image for correction as described above.

Alternatively, images for correction captured before and after image capturing through use of radiation may be used. In this case, by using images for correction captured before and after image capturing through use of radiation, a change in images for correction during image capturing may be expected to generate images for correction. They are generated by, for example, averaging images for correction captured before and after radiography, or complementing variations linearly or curvilinearly.

Since the time required for image capturing is short in the case of still image capturing, a change in images for correction before and after image capturing is small. In the case of kymography, the time required for image capturing is far longer than in still image capturing. Therefore, by using images for correction captured before and after image capturing as described above, correction is made considering variations during image capturing as well.

Upon sensing that the image capturing start signal has been turned off, and further, when image capturing after radiation emission and image capturing for acquiring images for correction described above are terminated, the image capturing device 3A turns off the readout instruction signal (see FIG. 16), and transmits a remaining image (untransmitted captured image) left in the memory of the image capturing device 3A to the console 4 (step S32). When transmission of the remaining image is completed, the image capturing device 3A transmits a remaining image transmission completion signal to the console 4 (step S33).

Upon sensing that the image capturing start signal has been turned off, the console 4 starts an operation of confirming the transmitted captured images.

Upon receipt of the remaining image transmission completion signal, the console 4 transmits an image deletion signal that instructs deletion of images to the image capturing device 3A (step S34).

Control may be exerted to transmit the image deletion signal after the operation of confirming the captured images is completed, and after it is confirmed that all of the transmitted images have no problem.

On that occasion, “image capturing terminated” may be displayed on the display 43 of the console 4 (step S35).

Upon receipt of the image deletion signal, the image capturing device 3A deletes captured images stored in the memory (step S36). Accordingly, free space of the memory is ensured for next image capturing.

When the radiographer confirming that image capturing has been terminated (for example, visually recognizing the display of “image capturing terminated” displayed on the console 4, or the like) releases the second stage of the emission instruction switch 5 (step S37), the emission instruction switch 5 turns off the emission instruction signal (step S38), and further, the radiation controller 11 also turns off the emission instruction signal (step S39).

Thereafter, when the radiographer releases the first stage of the emission instruction switch 5 (step S40), the emission instruction switch 5 turns off the emission preparation signal (step S41), and further, the radiation controller 11 also turns off the emission preparation signal (step S42).

Upon sensing that the emission preparation signal has been turned off, the additional device 6 notifies the console 4 of the fact.

Upon receipt of the notification from the additional device 6, the console 4 turns off the image capturing preparation completion signal to cause the sequence state to transition to an emission preparation state.

Upon sensing that the emission instruction signal and emission preparation signal have been turned off, the additional device 6 transmits an image capturing termination signal indicating that image capturing has been terminated to the image capturing device 3A and the console 4 (steps S43, S44).

Upon receipt of the image capturing termination signal, the image capturing device 3A transmits a standby signal to the console 4 (step S45).

Upon receipt of the standby signal, the console 4 monitors whether image capturing is performed again or another type of image capturing is performed for a predetermined period, and in a case where the predetermined period elapses without image capturing performed again or another type of image capturing performed, turns off the sequence start signal to cause the sequence state to transition to a waiting state of waiting for an image capturing instruction.

In this manner, a series of image capturing operations are terminated.

The system 100A according to the present embodiment operates as described above, and accordingly, kymography of repeatedly capturing a plurality of still images for a short time is performed.

Variation 1: Count Number of Already Captured Images in Image Capturing Device 3A

The above-described embodiment has shown the example in which the additional device 6 counts the number of times of transmitting the timing signal, and in the case where the counted number of times of outputting the timing signal reaches the maximum number of captured images N, it is determined that the maximum number of captured images N has been reached, whilst a device configuration may be adopted in which the number of times that the image capturing device 3A receives the timing signal after transmission of the emission start signal, or the number of times of receiving the timing signal and performing readout of the image capturing device 3A, or the number of times of performing readout of the image capturing device 3A and storing an image or transmitting the image to the console 4 is counted, and a determination is made depending on whether they have reached the maximum number of captured images N previously set.

Variation 2: Image Capturing Permission Depending on State of Image Capturing Device

The remaining amount of power and remaining amount of memory of the image capturing device 3A may be referred to when connecting the image capturing device 3A, the console 4, and the additional device 6 or when starting image capturing, and it may be determined whether designated kymography can be performed to the end.

Depending on the determination result, the fact that image capturing is allowed may be displayed if image capturing is allowed.

Depending on the determination result, the fact that image capturing is not allowed may be displayed if image capturing is not allowed.

Variation 3

The timing signal or/and information signal may be transmitted to the image capturing device 3A by a wired connection or a wireless connection.

In the case of transmitting the timing signal by a wireless connection, timing information through use of the Timing Synchronization Function (hereinafter, TSF) defined in the wireless communication standard IEEE802.11 or a signal generated based on this timing information may be used as the timing signal.

Effects

In the system 100A according to the present embodiment as described above, by connecting the additional controller 61 to the radiation control device 1 in the conventional system 100 (see FIG. 1) that performs emission of pulsed radiation only once in response to an instruction to emit radiation once or emits radiation only for a period in which a user is pressing the emission instruction switch 5, the radiation control device 1 continues outputting the emission signal for a predetermined time previously set in response to acquisition of a single emission instruction signal (sensing of turn-on). This enables image capturing through use of the image capturing device 3A in which still images (frames) are repeatedly generated multiple times for a short time, that is, kymography, to be performed.

The conventional system 100 has been spread widely as a radiation device that captures a simple still image. Thus, a medical institution using the conventional system 100 easily alters the conventional system 100 including an existing radiation generation device to be adaptable to kymography merely by adding the image capturing device 3A and the additional device 6, without upgrade to an expensive radiation generation device.

### Second Embodiment

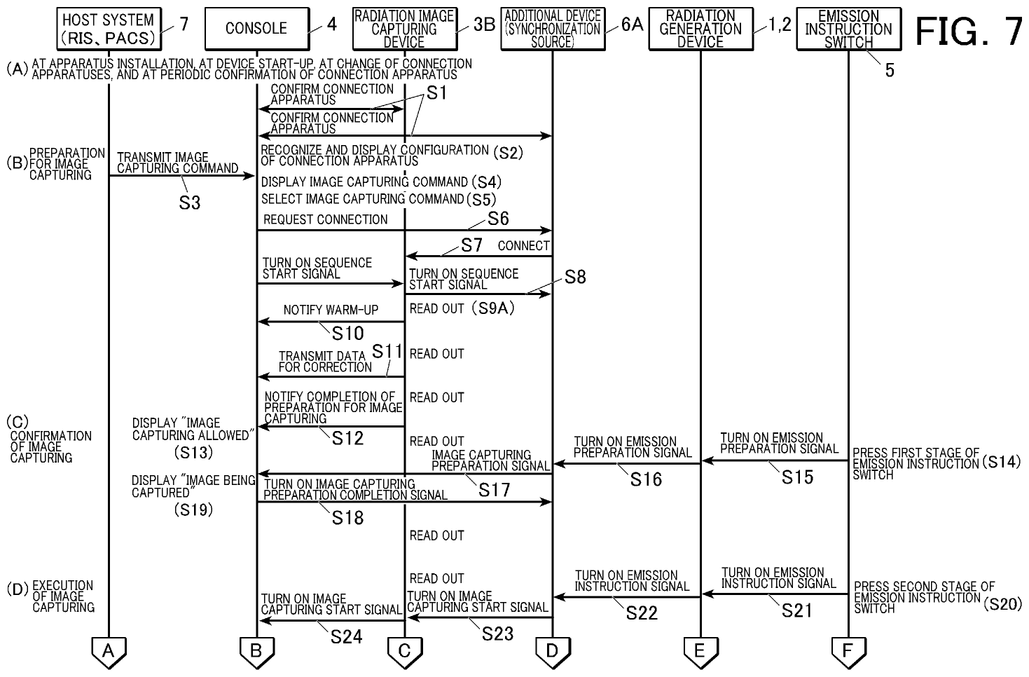

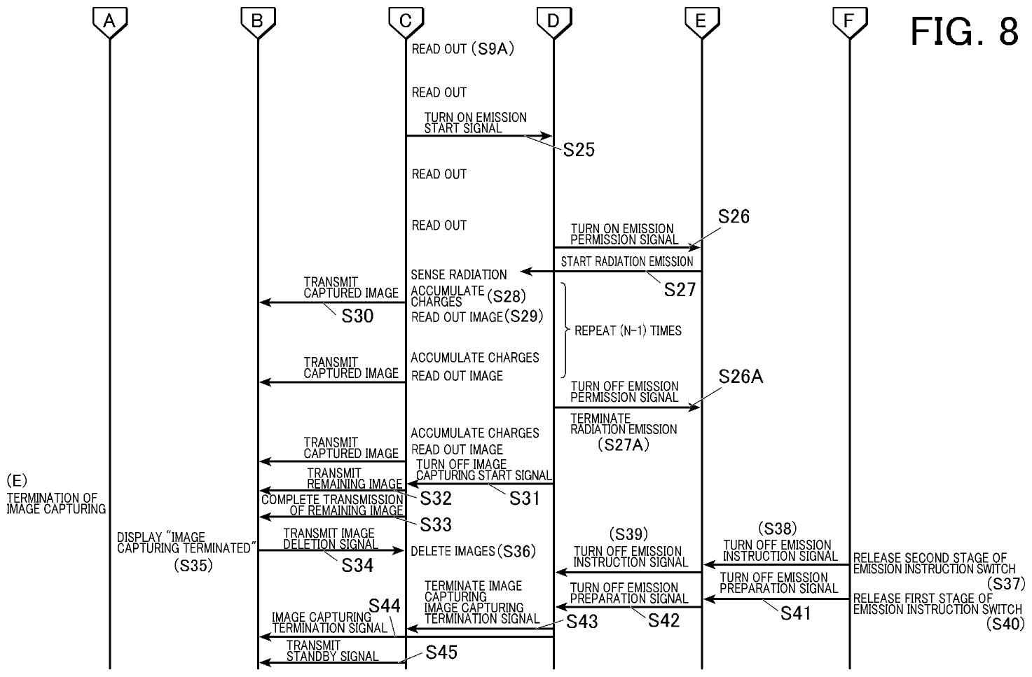

A second embodiment of the present invention will now be described with reference to FIG. 2, FIG. 7, and FIG. 8. Components equivalent to those of Conventional technology 1 and the first embodiment described above will be given identical reference characters, and description thereof will be omitted. Various variation patterns described in the first embodiment are also applicable to the present embodiment.

System Configuration

First, a system configuration of a radiography system (hereinafter, a system 100B) according to the present embodiment will be described.

The system 100B according to the present embodiment includes a radiation image capturing device instead of the cassette 3 of the conventional system 100 (see FIG. 1), and further includes the image capturing device control console 42 and an additional device, as shown in FIG. 2, similarly to the first embodiment. However, the system 100B according to the present embodiment is different from the first embodiment in terms of the configurations of the radiation image capturing device (hereinafter, an image capturing device 3B) and the additional device 6A.

Specifically, the image capturing device 3B is triggered by the fact that radiation from the radiation generation device has been sensed to start accumulation of charges and readout of an image, rather than performing accumulation of charges and readout of an image based on the timing signal from the additional device 6 as in the image capturing device of the above-described embodiment, and thereafter, repeats accumulation of charges and readout of an image at an image capturing frame rate previously set.

The image capturing device 3B starts counting the number of captured images when accumulation of charges is started, and when the counted number reaches the number of captured images previously set, stops repeating accumulation of charges and readout of an image.

The additional device 6A does not transmit the timing signal.

When the emission permission signal is turned on, the additional device 6A starts counting the elapsed time, and when the elapsed time reaches an image capturing time previously set, turns off the image capturing permission signal.

Operations

Operations of the above-described system 100B will now be described. FIG. 7 and FIG. 8 are ladder charts representing operations of the system 100B according to the present embodiment.

The operations of the system 100B according to the present embodiment are common to those of the first embodiment from the start to step S8, as shown in FIG. 7.

Before step S10 (notify warm-up) is performed, the image capturing device 3B starts the readout operation (step S9A). Thereafter, the image capturing device 3B repeats the readout operation at a predetermined frame rate.

Thereafter, as shown in FIG. 8, when the additional device 6A turns on the emission permission signal being output to the radiation generation device (step S26), and the radiation generation device emits radiation to the image capturing device 3B, the image capturing device 3B senses the radiation, and starts step S28 (accumulate charges) and step S29 (read out image). Thereafter, step S28 and step S29 are repeated N-1 times at a predetermined frame rate.

Operations in and subsequent to step S30 (transmit/store captured image, or the like) while step S28 and step S29 are being repeated and step S31 after step S28 and step S29 are repeated N times are similar to those of the first embodiment.

Effects

In the system 100B according to the present embodiment as described above, by connecting the additional controller 61A to the radiation control device 1 in the conventional system 100 (see FIG. 1) that performs emission of pulsed radiation only once in response to an instruction to emit radiation once or emits radiation only for a period in which a user is pressing the emission instruction switch 5, the radiation control device 1 continues outputting the emission signal for a predetermined time previously set in response to acquisition of a single emission instruction signal (sensing of turn-on), similarly to the first embodiment. This enables image capturing through use of the image capturing device 3B in which still images (frames) are repeatedly generated multiple times for a short time, that is, kymography, to be performed.

The conventional system 100 has been spread widely as a radiation device that captures a simple still image. Thus, a medical institution using the conventional system 100 easily alters the conventional system 100 including an existing radiation generation device to be adaptable to kymography merely by adding the image capturing device 3B and the additional device 6A, without upgrade to an expensive radiation generation device.

Wireless communication uses a best-effort packet transmission technology. Therefore, if the timing signal is transmitted from the additional device 6 to the image capturing device 3A through wireless communication when image capturing is performed in response to the timing signal from the additional device 6 as in the first embodiment, the time at which the signal arrives varies in some cases. Thus, it is difficult to use the timing signal transmitted wirelessly for controlling the image capturing timing

In clinical practice, there is a demand for performing image capturing immediately at a radiographer's desired timing.

Through use of the system 100B according to the present embodiment, kymography is started by simple control without exchanging the timing signal or the like between the image capturing device 3B and the radiation generation device, which enables image capturing to be started immediately at a radiographer's desired timing

Conventional Technology 2

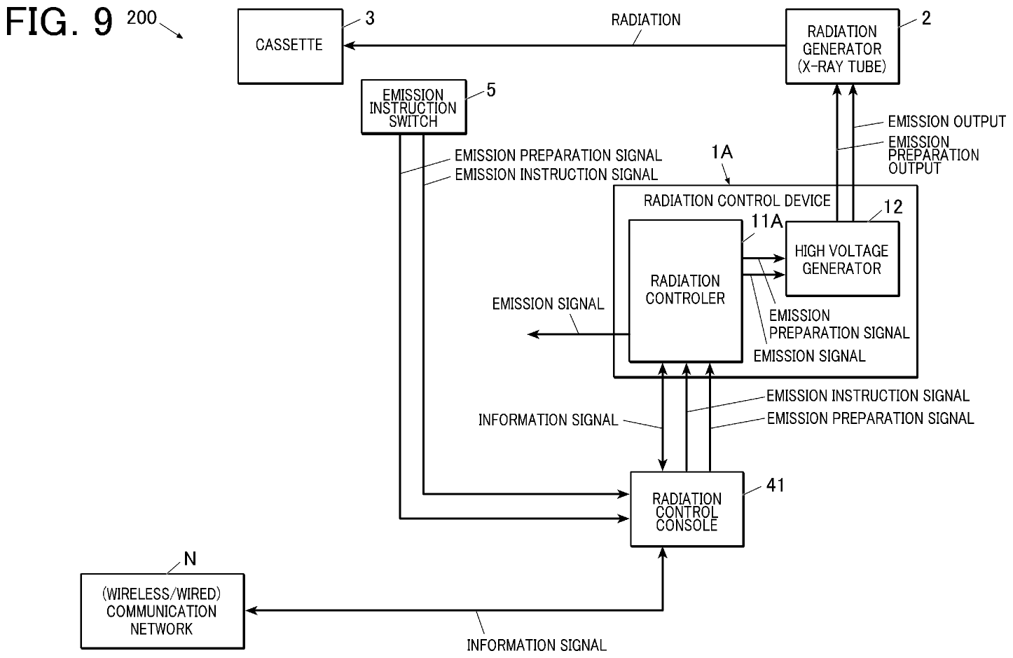

Conventional technology 2 on which radiography systems (details of which will be described later) according to third and fourth embodiments of the present invention are based will now be described with reference to FIG. 9. Components equivalent to those of Conventional technology 1 above will be given identical reference characters, and description thereof will be omitted.

System Configuration

First, a schematic configuration of a radiography system (hereinafter, a conventional system 200) according to Conventional technology 2 will be described. FIG. 9 is a block diagram representing a schematic configuration of the conventional system 200.

The conventional system 200 is different from the conventional system 100 in terms of the configuration of a radiation controller 11A included in a radiation control device 1A, as shown in FIG. 9, for example.

Specifically, upon sensing that the emission preparation signal and the emission instruction signal from the radiation control console 41 have been turned on, the radiation controller 11 of the conventional system 100 outputs them to an external apparatus, whilst the radiation controller 11A of the conventional system 200 does not have such a configuration.

The radiation controller 11 of the conventional system 100 also receives the emission permission signal from an external apparatus, whilst the radiation controller 11A of the conventional system 200 neither has such a configuration.

Operations

Operations of the conventional system 200 will now be described.

Emission Preparation Operation

When the first stage of the emission instruction switch 5 is pressed by the radiographer, the emission instruction switch 5 turns on the emission preparation signal to be output to the radiation controller 11A via the radiation control console 41.

Upon sensing that the emission preparation signal has been turned on, the radiation controller 11A turns on the emission preparation signal to be output to the high voltage generator 12.

Although FIG. 9 does not illustrate output of the emission preparation signal from the radiation controller 11A to an external apparatus, the emission preparation signal to the external apparatus may be output in a case of operating in cooperation with the external apparatus.

Upon sensing that the emission preparation signal has been turned on, the high voltage generator 12 outputs an emission preparation output to the radiation generator 2.

Upon receipt of the emission preparation output, the radiation generator 2 starts preparation for emitting radiation.

In the case of using a rotary anode as the anode, the operation such as rotating the rotary anode, for example, is performed.

It is considered that the radiation generation device (the radiation controller 11A, the high voltage generator 12, and the radiation generator 2) configured in this manner operates in the pulse emission mode in some cases, and operates in the continuous emission mode in other cases, similarly to the radiation generation device of the first embodiment.

Depending on the types of the radiation controller 11A, the high voltage generator 12, and the radiation generator 2, the radiation generation device is operable only in either the pulse emission mode or the continuous emission mode in some cases, and is adaptable to both the modes in other cases.

Emission Operation

Subsequently, when the second stage of the emission instruction switch is pressed by the radiographer, the emission instruction switch 5 turns on the emission instruction signal to be output to the radiation controller 11A via the radiation control console 41.

Although FIG. 9 does not illustrate output of the emission instruction signal from the radiation controller 11A to an external apparatus, the emission instruction signal to the external apparatus may be output in a case of operating in cooperation with the external apparatus.

Since the emission permission signal is not received from the external apparatus in Conventional technology 2, control of transmitting the emission signal in the case where the emission instruction signal and the emission permission signal are completed is not exerted. Thus, the radiation controller 11A turns on the emission signal to be output to the high voltage generator 12 merely by sensing that the emission instruction signal has been turned on.

Upon sensing that the emission signal has been turned on, the high voltage generator 12 applies a high voltage necessary for radiation emission in the radiation generator 2 to the radiation generator 2 as an emission output.

When the high voltage is applied from the high voltage generator 12, the radiation generator 2 generates radiation in accordance with the applied voltage.

The generated radiation is adjusted in terms of the direction of emission, area, radiation quality, and the like by a controller not shown such as a collimator, and is emitted to the subject and the cassette 3 behind the subject. Radiation partly passes through the subject to enter the cassette 3.

When radiation enters the cassette 3, a radiation image is formed on a stored film or fluorescent plate.

In order to prevent emission from being performed before the rotation of the rotary anode reaches a sufficient speed, the radiation controller 11A may be configured not to transmit the emission signal even upon sensing that the emission instruction signal has been turned on, until a predetermined waiting time elapses upon sensing that the emission preparation signal has been turned on as described above, similarly to Conventional technology 1 above.

In this manner, in radiography through use of the conventional system 200, only a single radiation image (still image) of the subject is captured based on a single image capturing manipulation, similarly to the case of using the conventional system 100.

As described above, in the case where the radiation generation device has only one of the pulse emission mode and the continuous emission mode, a device having the pulse emission mode and a device having the continuous emission mode are prepared respectively, and image capturing is performed in a desired mode using the radiation generation device corresponding to the desired mode.

In a case where the radiation generation device has both the pulse emission mode and the continuous emission mode, and switches between the modes in the radiation controller 11A or the high voltage generator 12 or switches between the modes by externally making an input or the like to the radiation controller 11A or the high voltage generator 12, image capturing is performed in a desired mode by selecting, on the radiation control console 41, a mode in which image capturing is to be performed when inputting image capturing conditions before image capturing, for example, and selecting the operation of the radiation controller 11A or the high voltage generator 12 before image capturing.

### Third Embodiment

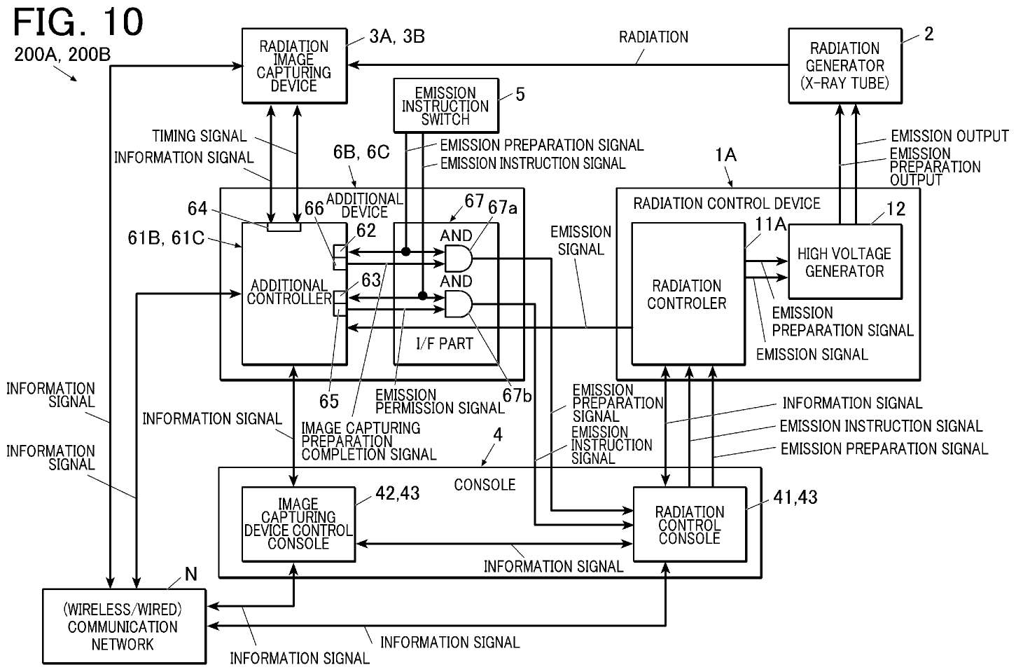

The third embodiment of the present invention will now be described with reference to FIG. 10 to FIG. 12. Components equivalent to those of Conventional technology 2 and the first and second embodiments described above will be given identical reference characters, and description thereof will be omitted. The various variation patterns described in the first and second embodiments are also applicable to the present embodiment.

Preposition, Background, and Problems

Some radiography systems have the radiation controller 11 that has an input part for inputting the emission permission signal from the outside, and transmits the emission signal in response to an emission instruction from the radiographer and emission permission from the outside as described in Conventional technology 1 above, while others have the radiation controller 11A that only has an input part for inputting the emission instruction signal from the outside, and captures a still image as described in Conventional technology 2 above.

The radiography system (hereinafter, a system 200A) according to the present embodiment enables continuous image capturing to be performed by applying the additional device 6B to such a radiation controller 11A.

System Configuration

First, a system configuration of the system 200A will be described. FIG. 10 is a block diagram representing a schematic configuration of the system 100 according to the first embodiment. Reference characters in parentheses in FIG. 10 belong to the fourth embodiment which will be described later.

The system 200A according to the present invention includes the image capturing device 3A instead of the cassette 3 of the conventional system 200 (see FIG. 9), and further includes the image capturing device control console 42 and the additional device 6B similar to those of the first embodiment, as shown in FIG. 10, for example.

The additional device 6B includes an additional controller 61B and an interface (hereinafter, the I/F part 67).

Although FIG. 10 illustrates the additional device 6B including the additional controller 61B and the I/F part 67 separately, they may be formed integrally.

The additional controller 61B has a third connector 66 in addition to the first acquisitor 62, the second acquisitor 63, the first connector 64, and the second connector 65 similar to those of the first embodiment.

The I/F part 67 has a first AND circuit 67a and a second AND circuit 67b.

The first acquisitor 62 is connected to one of input parts of the first AND circuit 67a, and the third connector 66 is connected to the other input part of the first AND circuit 67a.

The second acquisitor 63 is connected to one of input parts of the second AND circuit 67b, and the second connector 65 is connected to the other input part of the second AND circuit 67b.

In the system 100 according to the first embodiment, the emission instruction switch 5 is connected to the console 4, and the emission instruction switch 5 outputs the emission preparation signal and the emission instruction signal to the additional device 6 via the radiation control device 1, whilst in the system 200A according to the present embodiment, the emission instruction switch 5 that outputs the emission preparation signal and the emission instruction signal is directly connected to the additional device 6B.

The additional device 6B inputs the emission preparation signal and the emission instruction signal from the emission instruction switch 5 to the additional controller 61B and one input parts of the first and second AND circuits 67a and 67b of the I/F part 67, respectively. That is, the first acquisitor 62 acquires the emission preparation signal, and the second acquisitor 63 acquires the emission instruction signal, directly from the emission instruction switch 5.

A substrate or an apparatus provided with the emission instruction switch 5 may be connected to the I/F part 67, and the first and second acquisitors 62 and 63 may acquire the emission preparation signal and the emission instruction signal output from the emission instruction switch 5 via the substrate or apparatus.

The third connector 66 according to the present embodiment outputs the image capturing preparation completion signal to the first AND circuit 67a, and the second connector 65 outputs the emission permission signal to the second AND circuit 67b, and in a case where AND conditions are satisfied with the emission preparation signal and the emission instruction signal from the emission instruction switch 5 in the first and second AND circuits 67a and 67b, outputs the emission preparation signal and the emission instruction signal to the radiation controller 11 via the radiation control console 41.

That is, the second connector 65 according to the present embodiment is connectable to the radiation generation device via the I/F part 67. Thus, the I/F part 67 and the radiation control console 41 of the present embodiment constitute the relay in the present invention.

FIG. 9 and FIG. 10 show the radiography systems in which the emission preparation signal and the emission instruction signal to be input to the radiation controller 11A are input via the radiation control console 41. However, some radiography systems may not have a radiation control console. In such a case, radiation emission conditions are set by the image capturing device control console 42 and the radiation controller 11A performing information communication. In such a case, the emission preparation signal and the emission instruction signal are input to the radiation controller 11A.

In other radiography systems, although the radiation control console 41 is provided, the radiation control console 41 makes settings for the radiation emission conditions merely by performing information communication with the radiation controller 11A, and the emission preparation signal and the emission instruction signal are input to the radiation controller 11A.

Also in such a case, the configuration described in the present invention is implemented by causing the emission preparation signal and the emission instruction signal to be input to the radiation controller 11A without the intervention of the radiation control console 41.

FIG. 10 shows the example in which the emission preparation signal from the emission instruction switch 5 is also branched in the I/F part 67 to be input to the additional controller 61B and the first AND circuit 67a, and when AND conditions are satisfied with the image capturing preparation completion signal from the additional controller 61B, the emission preparation signal is output from the I/F part 67. However, rather than adopting such a configuration for the emission preparation signal, the emission preparation signal may be directly output to the radiation control console 41 and the radiation controller 11A from the emission instruction switch 5.

Although FIG. 10 illustrates the configuration in which the first connector 64 directly transmits/receives information and signals to/from the image capturing device 3A, the first connector 64 may be connectable to another device via a relay not shown that relays a signal.

Although FIG. 10 illustrates the case in which the first acquisitor 62, the second acquisitor 63, the first connector 64, the second connector 65, and the third connector 66 are provided separately, at least two of the first acquisitor 62, the second acquisitor 63, the first connector 64, the second connector 65, and the third connector 66 may be formed integrally (the respective components 62 to 66 may be shared).

Although illustration is omitted, the emission preparation signal and the emission instruction signal output from the additional device 6B may be directly input to the radiation controller 11A without the intervention of the radiation control console 41.

The additional controller 61B is different from the additional controller 61 according to the first embodiment in terms of programs to be executed, and may have a similar structure to that of the additional controller 61 according to the first embodiment. (Although the additional controller 61 according to the first embodiment also includes the third connector 66, illustration of which is omitted in FIG. 2, a controller similar to the additional controller 61 can also be used by not including a command for using the third connector 66 in the programs). Alternatively, the additional controller 61B limited to necessary functions may be used separately from the additional controller 61.

Upon sensing that the emission preparation signal from the emission instruction switch 5 has been turned on, the additional controller 61B turns on the image capturing preparation signal to be output to at least one of the image capturing device 3A and the console 4.

Upon sensing that the image capturing preparation completion signal from at least one of the console 4 and the image capturing device 3A has been turned on, the additional controller 61B turns on the image capturing preparation completion signal to be output to the other input part of the first AND circuit 67a of the I/F part 67.

Upon sensing that the emission instruction signal from the emission instruction switch 5 has been turned on, the additional controller 61B turns on the image capturing start signal to be output to at least one of the image capturing device 3A and the console 4.

Upon sensing that the emission start signal from at least one of the console 4 and the image capturing device 3A has been turned on, the additional controller 61B turns on the emission permission signal similar to that of the first embodiment to be output to the other input part of the second AND circuit 67b of the I/F part 67.

The additional controller 61B repeatedly outputs the timing signal (for example, a pulsed signal) similar to that of the first embodiment to the image capturing device 3A in a predetermined cycle.

For controlling the output period of the emission permission signal and the transmission timing of the timing signal in this manner, the additional controller 61B can have a timer similar to that of the first embodiment.

Operations

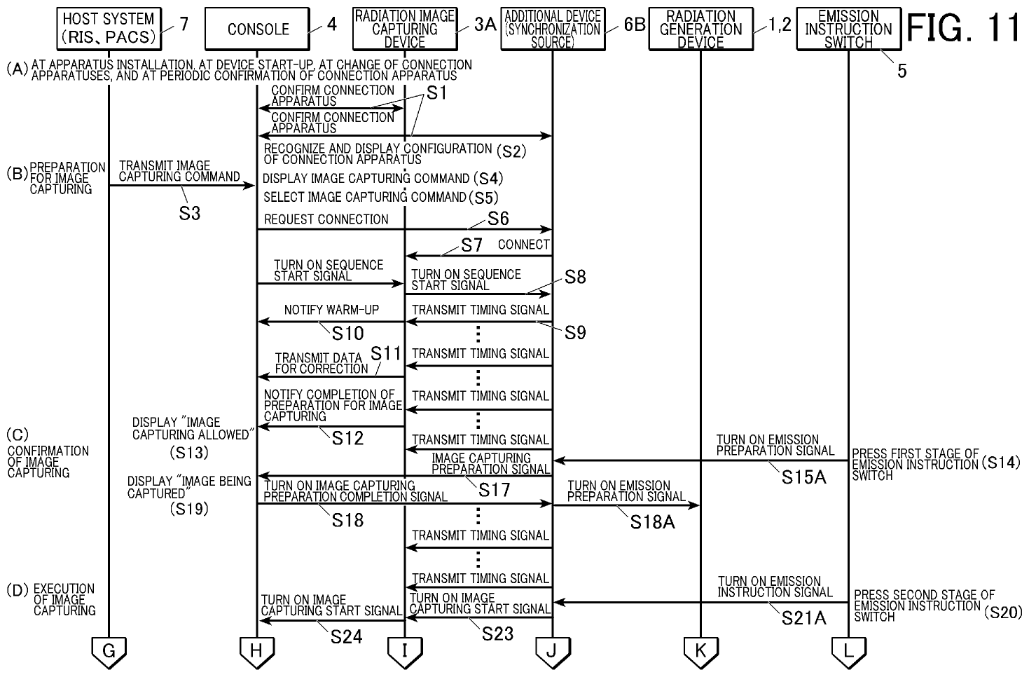

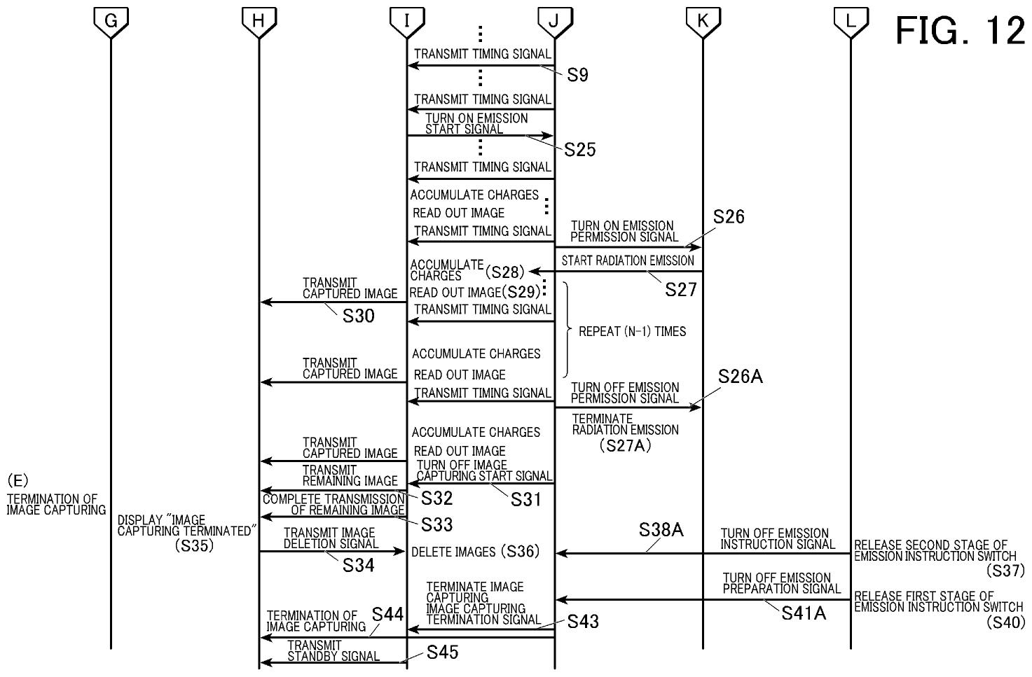

Operations of the system 200A will now be described. FIG. 11 and FIG. 12 are ladder charts representing the operations of the system 200A according to the present embodiment.

As shown in FIG. 11, operations for A) AT APPARATUS INSTALLATION, AT DEVICE START-UP, AT CHANGE OF CONNECTION APPARATUSES, AND AT PERIODIC CONFIRMATION OF CONNECTION APPARATUS (steps S1 and S2) and operations for B) PREPARATION FOR IMAGE CAPTURING (steps S3 to S13) are similar to those of the first embodiment.

C) Confirmation of Image Capturing (Preparation for Emission)

The additional device 6B continuously transmits the timing signal repeatedly to the image capturing device 3A, and the image capturing device 3A repeats the readout operation of the image capturing device 3A each time this timing signal is received.

When the radiographer terminates positioning of the subject, and presses the first stage of the emission instruction switch 5 (step S14), the emission instruction switch 5 turns on the emission preparation signal to be output to the additional device 6B (step S15A).

The emission preparation signal is input to the additional controller 61B and one of input parts of the first AND circuit 67a of the I/F part 67, respectively.

At this time, the additional controller 61B is connected to the other input part of the first AND circuit 67a. Thus, in a case where the image capturing preparation completion signal input to the other input part has not been turned on even if the emission preparation signal input from the emission instruction switch 5 to the one of the input parts of the first AND circuit 67a has been turned on, the emission preparation signal to be output from the first AND circuit 67a to the radiation control console 41 remains off.

Upon sensing that the emission preparation signal from the emission instruction switch 5 has been turned on, the additional controller 61B transmits the image capturing preparation signal that instructs preparation for image capturing to at least one of the console 4 and the image capturing device 3A (step S17).

Upon receipt of the image capturing preparation signal, at least one of the console 4 and the image capturing device 3A performs preparation for image capturing, and when preparation for image capturing is completed, turns on the image capturing preparation completion signal to be output to the additional device 6B (step S18).

Control Over Preparation for Image Capturing in External Apparatus

In a case where at least one of the console 4 and the image capturing device 3A has a connector for inputting the image capturing preparation completion signal indicating whether preparation for image capturing has been completed from an external apparatus although illustration is omitted, at least one of the console 4 and the image capturing device 3A may turn on the image capturing preparation completion signal in a case where it is sensed that the image capturing preparation completion signal from the external apparatus has been turned on.

Alternatively, although illustration is omitted, the additional device 6B or the additional controller 61B may be provided with a connector for outputting the image capturing preparation signal to the external apparatus, or a connector for inputting the image capturing preparation completion signal from the external apparatus.

This enables preparation for image capturing to be instructed from the additional device 6B or the additional controller 61B to the external apparatus, or enables completion of preparation for image capturing in the external apparatus to be sensed, and enables the image capturing preparation completion signal to be output to the I/F part also in response to completion of preparation for image capturing in the external apparatus.

By sensing that the image capturing preparation completion signal has been turned on, the additional device 6B recognizes that at least one of the console 4 and the image capturing device 3A or the external apparatus is in a state allowed to perform image capturing, and by exerting control so as to emit radiation after the image capturing preparation completion signal is turned on, the danger that radiation is emitted in the state where at least one of the console 4 and the image capturing device 3A or the external apparatus is not allowed to perform image capturing to uselessly expose the subject to radiation is eliminated reliably.

Upon sensing that the image capturing preparation signal has been turned on, or upon start of the image capturing preparation operation, or upon completion of the image capturing preparation operation, at least one of the console 4 and the image capturing device 3A turns on a signal indicating whether the image capturing preparation signal has been received, or a signal indicating whether the image capturing preparation operation has been started, or the image capturing preparation completion signal indicating whether the image capturing preparation operation has been completed, to be output to the additional device 6B (step S18).

Upon sensing that the image capturing preparation completion signal has been turned on, the additional device 6B turns on the image capturing preparation completion signal to be output to the other input part of the first AND circuit 67a of the I/F part 67.

At this time, since the emission preparation signal from the emission instruction switch 5 to be input to the first AND circuit 67a of the I/F part 67 and the image capturing preparation completion signal from the additional controller 61B are both turned on, the first AND circuit 67a turns on the emission preparation signal to be output to the radiation control console 41.

Upon sensing that the emission preparation signal has been turned on, the radiation control console 41 turns on the emission preparation signal to be output to the radiation controller 11A (radiation generation device). That is, the additional device 6B turns on the emission preparation signal to be transmitted to the radiation generation device via the radiation control console 41 (step S18A).

Upon sensing that the emission preparation signal has been turned on, the radiation generation device (the radiation controller 11A, the high voltage generator 12, and the radiation generator 2) performs preparation for radiation emission similar to that of the first embodiment.

Herein, the case in which the additional device 6B transmits the emission preparation signal to the radiation controller 11A upon confirming that preparation for image capturing in the image capturing device 3A and the console 4 has been completed (upon receipt of the image capturing preparation completion signal) has been described, whilst the emission preparation signal may be transmitted to the radiation controller 11A concurrently with transmission to the image capturing device 3A and the console 4 without confirming completion of preparation for image capturing in the image capturing device 3A and the console 4.

In this case, the first AND circuit 67a of the I/F part 67 is unnecessary, and the emission preparation signal received from the emission instruction switch 5 may be distributed to the console 4, the image capturing device 3A, the radiation control console 41, or the radiation controller 11A.

D) Execution of Image Capturing

Subsequently, when the radiographer presses the second stage of the emission instruction switch 5 (step S20), the emission instruction switch 5 turns on the emission instruction signal to be output to the additional device 6B (step S21A).

At this time, the additional device 6B continuously transmits the timing signal repeatedly to the image capturing device 3A, and the image capturing device 3A repeats the readout operation each time this timing signal is received.

The emission instruction signal is input to each of the additional controller 61B and one of the input parts of the second AND circuit 67b of the I/F part 67.

At this time, the additional controller 61B is connected to the other input part of the second AND circuit 67b. Thus, in a case where the emission permission signal is not input to the other input part even if the emission instruction signal input from the emission instruction switch 5 to the one input part of the second AND circuit 67b has been turned on, the emission instruction signal output from the second AND circuit 67b to the radiation control console 41 remains off.

Upon sensing that the emission instruction signal from the emission instruction switch 5 has been turned on, the additional device 6B turns on the image capturing start signal to be output to at least one of the console 4 and the image capturing device 3A (steps S23, S24).

Upon sensing that the image capturing start signal has been turned on, the image capturing device 3A is triggered by the termination of the readout operation being performed by itself at that point of time to turn on the emission start signal to be output to the additional device 6B, as shown in FIG. 12, for example (step S25).

Upon sensing that the emission start signal from the image capturing device 3A has been turned on, the additional controller 61B determines that the image capturing device 3A is in a state allowed to perform image capturing, and turns on the emission permission signal to be output from the additional controller 61B to the I/F part 67.

At this time, the emission instruction signal from the emission instruction switch 5 and the emission permission signal from the additional controller 61B, to be input to the second AND circuit 67b of the I/F part 67, are both turned on, and thus, the second AND circuit 67b turns on the emission instruction signal being output to the radiation controller 11A via the radiation control console 41 (step S26).

The operations in the latter half (steps S27 to S30) of “D: EXECUTION OF IMAGE CAPTURING” and operations in the first half (steps S31 to S36) of “E: TERMINATION OF IMAGE CAPTURING” are similar to those of the first embodiment.

Termination of Image Capturing

When the radiographer confirming that image capturing has been terminated releases the second stage of the emission instruction switch 5 (step S37), the emission instruction switch 5 turns off the emission instruction signal (step S38A). Then, the image capturing device 3A turns off the image capturing start signal.

Thereafter, when the radiographer releases the first stage of the emission instruction switch 5 (step S40), the emission instruction switch 5 turns off the emission preparation signal (step S41A).

Steps S43 to S45 are similar to those of the first embodiment.

In this manner, a series of image capturing operations are terminated.

The system 200A according to the present embodiment operates as described above, and accordingly, kymography of repeatedly capturing a plurality of still images for a short time is performed, similarly to the system 100A according to the first embodiment.

Effects

In the system 200A according to the present embodiment as described above, by connecting the additional controller 61B to the radiation control device 1A in the conventional system 200 (see FIG. 9) that emits pulsed radiation only once in response to an instruction to emit radiation once or emits radiation only for a period in which a user is pressing the emission instruction switch 5, the radiation control device 1A continues outputting the emission signal for a predetermined time previously set in response to an instruction to emit radiation once (pressing of the second stage of the emission instruction switch 5). This enables image capturing through use of the image capturing device 3A in which still images (frames) are repeatedly generated multiple times for a short time, that is, kymography, to be performed.

The conventional system 200 has been spread widely as a radiation device that captures a simple still image. Thus, a medical institution using the conventional system 200 easily alters the conventional system 200 including an existing radiation generation device to be adaptable to kymography merely by adding the image capturing device 3A and the additional device 6B, without upgrade to an expensive radiation generation device.

In the system 200A according to the present embodiment, the additional device 6B may be divided into the additional controller 61B and the I/F part 67, and the additional controller 61B may have the same structure as the additional controller 61 of the first embodiment (the only difference resides in programs stored therein). This enables both the additional device 6 of the first embodiment and the additional device 6B of the second embodiment to be produced (enables both the conventional system 100 and the conventional system 200 to be altered) using common components, without increasing the types of devices.

In the second embodiment, the configuration of adding the additional device 6B to the conventional system 200 (see FIG. 9) so that kymography can be performed has been described. However, the embodiments of the present invention are not limited to this, but the additional device 6B of the second embodiment can also be added to the conventional system 100 (see FIG. 1) so that kymography can be performed, for example.

By causing the emission permission signal to be input to the radiation controller 11 of the conventional system 100 to remain on all the time, for example, the conventional system 100 is configured as the radiography system according to the present invention.

Such a configuration enables kymography to be performed by adding the additional device to various radiography systems.

### Fourth Embodiment

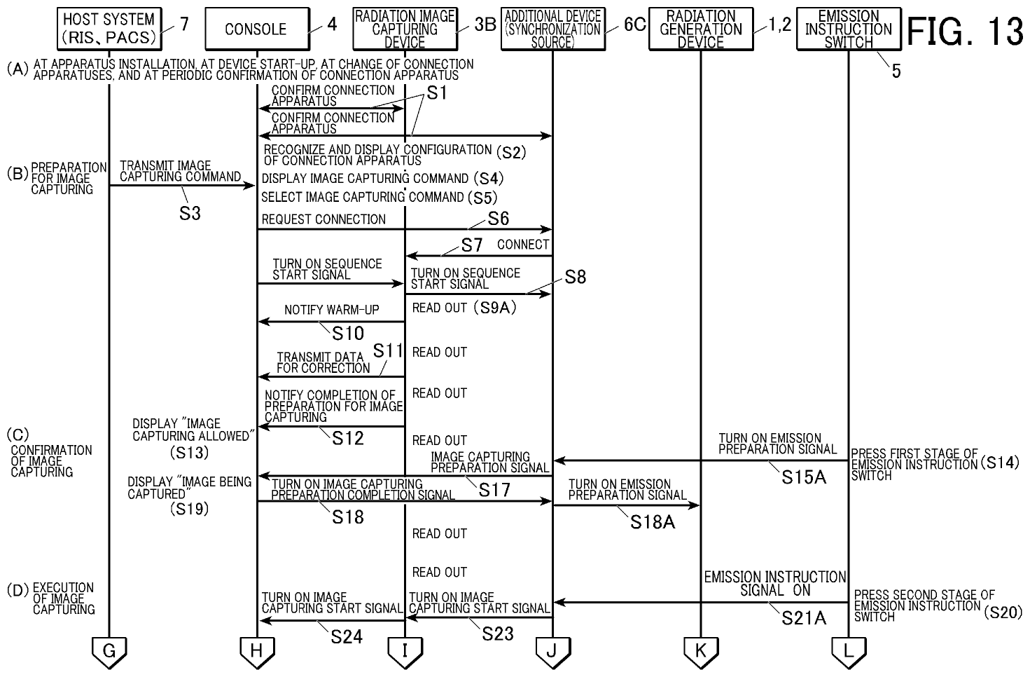

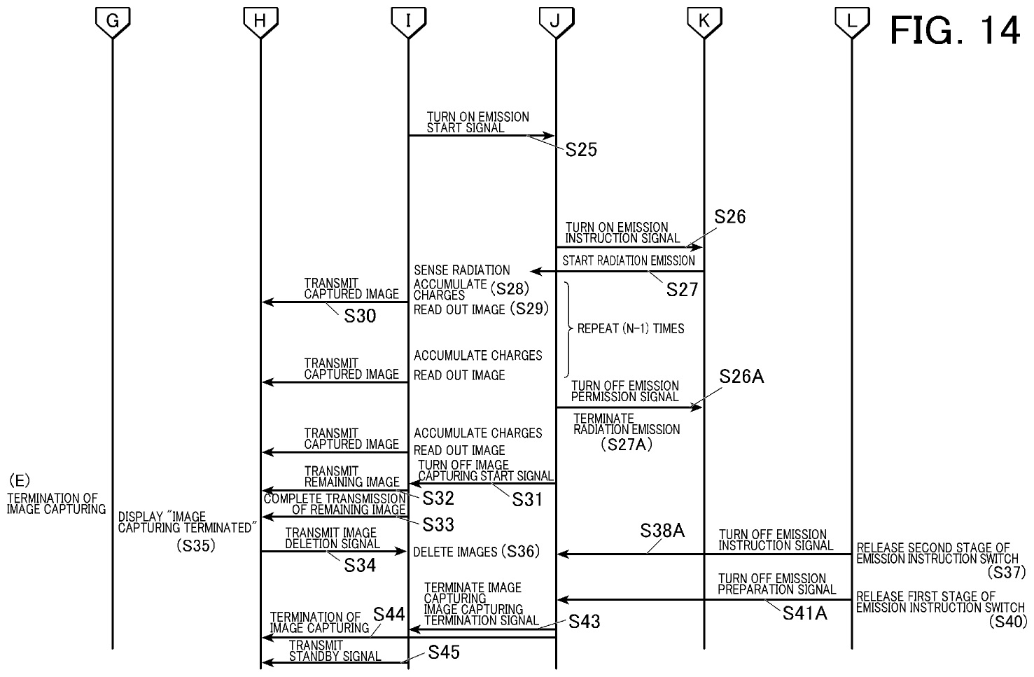

The fourth embodiment of the present invention will now be described with reference to FIG. 10, FIG. 13, and FIG. 14. Components equivalent to those of Conventional technology 2 and the third embodiment described above will be given identical reference characters, and description thereof will be omitted. The various variation patterns described in the third embodiment are also applicable to the present embodiment.

System Configuration

First, a system configuration of a radiography system (hereinafter, a system 200B) according to the present embodiment will be described.

The system 200B according to the present embodiment includes a radiation image capturing device instead of the cassette 3 of the conventional system 200 (see FIG. 9), and further includes the image capturing device control console 42 and an additional device as shown in FIG. 10, similarly to the third embodiment. However, the system 200B according to the present embodiment is different from the first embodiment in terms of the configuration of the radiation image capturing device (hereinafter, the image capturing device 3B) and an additional device 6C.

Specifically, the image capturing device 3B is triggered by sensing radiation from the radiation generation device to start accumulation of charges and readout of an image, and thereafter, repeats accumulation of charges and readout of an image at an image capturing frame rate previously set, similarly to the second embodiment.

When accumulation of charges is started, the image capturing device 3B starts counting the number of captured images, and when the counted number reaches the number of captured images previously set, stops repeating accumulation of charges and readout of an image.

The additional device 6C does not transmit the timing signal, similarly to the second embodiment.

When the emission permission signal is turned on, the additional device 6C starts counting the elapsed time, and when the elapsed time reaches an image capturing time previously set, turns off the image capturing permission signal.

Operations

Operations of the system 200B will now be described. FIG. 13 and FIG. 14 are ladder charts representing the operations of the system 200B according to the present embodiment.

The operations of the system 200B according to the present embodiment are common to those of the third embodiment from the start to step S8, as shown in FIG. 13.

Before step S10 (notify warm-up) is performed, the image capturing device 3B starts the readout operation (step S9A). Thereafter, the image capturing device 3B repeats the readout operation at a predetermined frame rate.

Thereafter, as shown in FIG. 14, the additional device 6C turns on the emission permission signal being output to the radiation generation device (step S26), and when the radiation generation device emits radiation to the image capturing device 3B (step S27), the image capturing device 3B senses the radiation, and starts step S28 (accumulate charges) and step S29 (read out image). Thereafter, step S28 and step S29 are repeated N-1 times at a predetermined frame rate.

Operations in and subsequent to step S30 (transmit/store captured image, or the like) in the middle of repeat of step S28 and step S29 and step S31 after step S28 and step S29 are repeated N times are similar to those of the third embodiment.

Effects

In the system 200B according to the present embodiment as described above, by connecting the additional controller 61C to the radiation control device 1A in the conventional system 200 (see FIG. 9) that emits pulsed radiation only once in response to an instruction to emit radiation once or emits radiation only for a period in which a user is pressing the emission instruction switch 5, the radiation control device 1A continues outputting the emission signal for a predetermined time previously set in response to acquisition of a single emission instruction signal (sensing of turn-on). This enables image capturing through use of the image capturing device 3B in which still images (frames) are repeatedly generated multiple times for a short time, that is, kymography, to be performed.

The conventional system 200 has been spread widely as a radiation device that captures a simple still image. Thus, a medical institution using the conventional system 200 easily alters the conventional system 200 including an existing radiation generation device to be adaptable to kymography merely by adding the image capturing device 3B and the additional device 6C, without upgrade to an expensive radiation generation device.

As described above, wireless communication uses a best-effort packet transmission technology. Therefore, if the timing signal is transmitted from the additional device 6C to the image capturing device 3A through wireless communication when image capturing is performed in response to the timing signal from the additional device 6 as in the first embodiment, the time at which the signal arrives varies in some cases. Thus, it is difficult to use the timing signal transmitted wirelessly for controlling the image capturing timing.

In clinical practice, there is a demand for performing image capturing immediately at a radiographer's desired timing.

Through use of the system 200B according to the present embodiment, kymography is started by simple control without an exchange of the timing signal or the like between the image capturing device 3B and the radiation generation device, which enables image capturing to be started immediately at a radiographer's desired timing

Transition of Sequence State

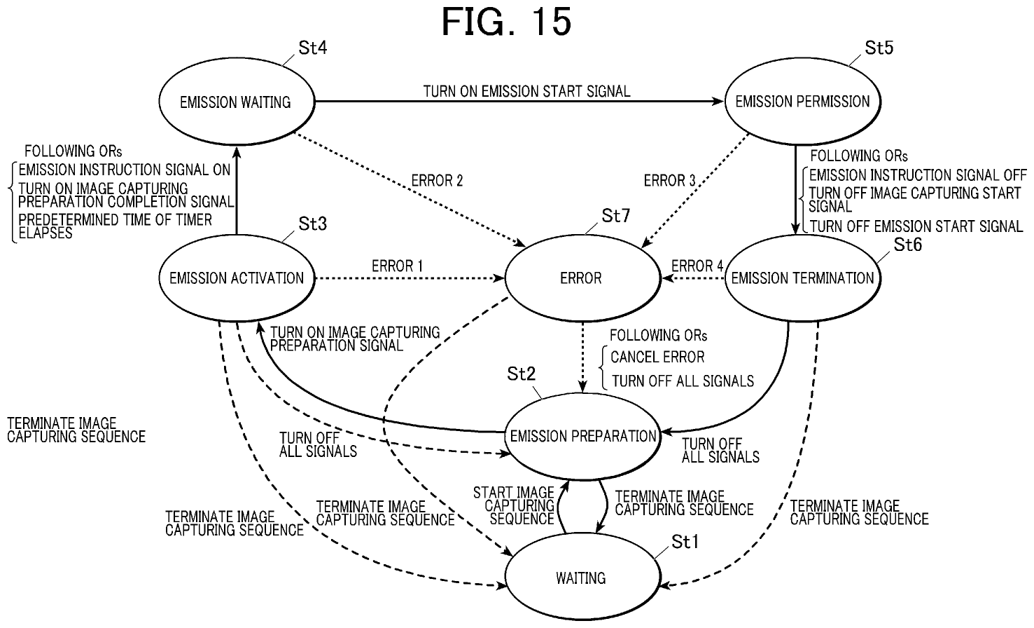

A sequence state transitioning operation in the systems 100A, 100B, 200A, and 200B according to the first to fourth embodiments (hereinafter, the system 100A or the like) will now be described with reference to FIG. 15 and FIG. 16.

Preposition, Background, and Problems

The system 100A or the like according to the first to fourth embodiments does not perform image capturing correctly if respective devices being connected operate in the correct order.

Even in a case where an error not intended by the radiographer, such as noise to a signal line or disconnection of a signal line, has occurred, it is necessary to terminate image capturing safely and to prevent unintended radiation emission or the like from occurring.

Operations

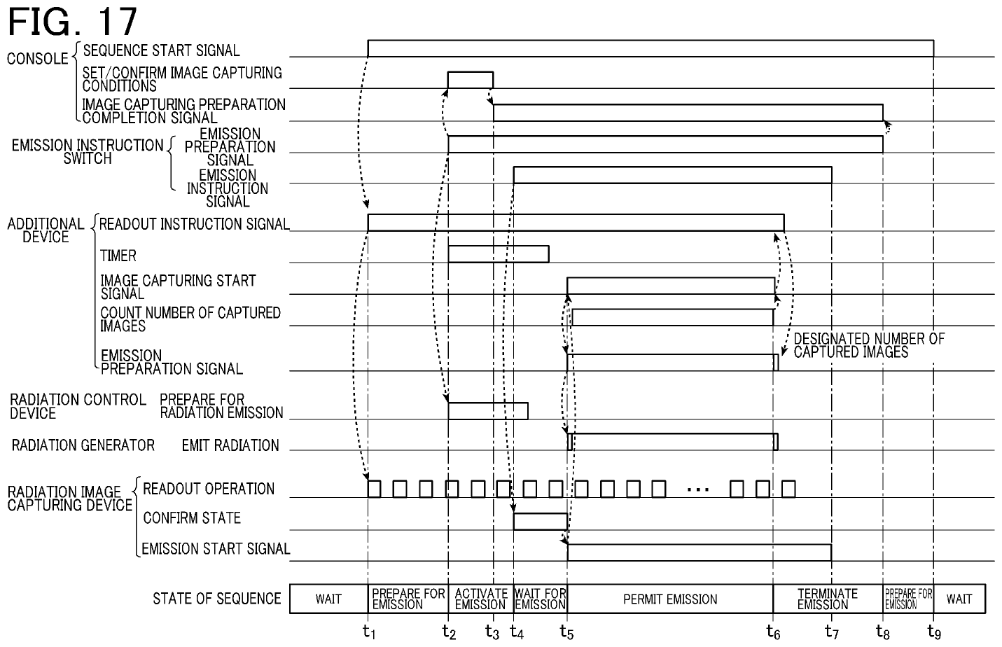

First, operations of the system 100A or the like will now be described. FIG. 15 is a state transition diagram of the system 100A or the like, FIG. 16 is a timing chart representing the operations of the systems 100A and 200A according to the first and third embodiments, and FIG. 17 is a timing chart representing the operations of the systems 100B and 200B according to the second and fourth embodiments.

The system 100A or the like according to the present embodiment is initially in a waiting state St1 in which an image capturing start instruction has not been received from the radiographer, as shown in FIG. 15.

Thereafter, when the console 4 receives an image capturing command from the host system 7 such as RIS or HIS, and the radiographer selects the image capturing command, the console 4 turns on the sequence start signal to be output to the image capturing device 3A/3B and the additional device 6/6A/6B/6C (hereinafter, the additional device 6 or the like) (t1), as shown in FIG. 16 and FIG. 17.

Then, the image capturing device 3A/3B as well as the additional device 6 or the like start preparation for image capturing. Accordingly, the system 100A or the like transitions to an emission preparation state St2 as shown in FIG. 15.

In the emission preparation state St2, the additional device 6 or the like of the system 100A/200A repeatedly transmits the timing signal to the image capturing device 3A at a predetermined interval, and the image capturing device 3A repeats the readout operation each time this timing signal is received to repeatedly perform the reset operation of removing charges accumulated in the image capturing device 3A, as shown in FIG. 16.

The image capturing device 3B of the system 100B/200B automatically repeats the readout operation to repeatedly perform the reset operation of removing charges accumulated in the image capturing device 3B, as shown in FIG. 17.

The readout operation performed herein is the same as the operation when acquiring a captured image. However, an image acquired by the reset operation is one generated in the emission preparation state St2 in which radiation is not emitted, and thus, may be stored in the memory of the image capturing device 3A/3B or transmitted to the console 4, or may be deleted without storage or transmission.

At least some of images acquired by this reset operation represent properties of respective pixels of the image capturing device 3A/3B or images of the image capturing device 3A/3B, and thus, may be stored in the image capturing device 3A/3B as images for correction for correcting a captured image or may be transmitted to the console 4, for example.

As an image for correction, at least one of a plurality of images acquired by repeating the reset operation may be used, or an average of signal values of corresponding pixels in a plurality of images or a complemental expected value in the temporal direction may be calculated to be used as an image for correction.

A method of correcting a captured image includes subtracting each of signal values of respective pixels of an image for correction from an image obtained by emitting radiation

The timing signal may be transmitted to the image capturing device 3A in a state other than the emission preparation state St2, and a reset operation instruction signal may be turned on when a transition is made to the emission preparation state St2, and the image capturing device 3A may perform the reset operation only in a case where the reset operation instruction signal has been turned on.

The radiographer sets image capturing conditions and the like using the image capturing device control console 42 or the radiation control console 41, and after performing positioning of the subject, starts the image capturing operation.

Specifically, as shown in FIG. 16 and FIG. 17, the emission instruction switch 5 is manipulated to turn on the emission preparation signal to be transmitted to the console 4 (t2). Then, the system 100A or the like transitions to an emission activation state St3, as shown in FIG. 15.

In the emission activation state St3, the console 4 confirms the state of the radiation control device 1, the image capturing device 3A/3B, and the additional device 6 or the like, and when determining that they are in the state allowed to perform image capturing, turns on the image capturing preparation completion signal to be transmitted to the additional device 6 or the like (t3), as shown in FIG. 15.

The console 4 may confirm whether image capturing conditions set in the radiation control console 41 and image capturing conditions set in the image capturing device control console 42 are equal, and if they are different, may display that they are different.

In the case where the image capturing conditions set in the radiation control console 41 and the image capturing conditions set in the image capturing device control console 42 are different, control may be exerted not to proceed into a subsequent image capturing sequence.

Control may be exerted not to allow the image capturing conditions set in the image capturing device control console 42 and the radiation control console 41 to be changed while the image capturing preparation completion signal is maintained in the on state.

Upon sensing that the emission preparation signal has been turned on, the radiation control device 1 starts preparation for radiation emission (t2). This is an operation such as starting rotation of the rotary anode of the radiation generator 2, for example.

Upon sensing that the emission preparation signal has been turned on, the additional device 6 or the like starts counting with a set timer (t2).

As will be described later in detail, this prevents a transition from being made to an emission waiting state St4 which will be described later until the count of this timer goes over a predetermined waiting time even if the radiographer presses the second stage of the emission instruction switch 5 (turns on the emission instruction signal).

Thereafter, the radiographer presses the second stage of the emission instruction switch 5 to turn on the emission instruction signal (t4). Although FIG. 16 and FIG. 17 illustrate the case in which the emission instruction signal is turned on after the image capturing preparation completion signal is turned on, whilst the emission instruction signal may be turned on before the image capturing preparation completion signal is turned on.

When the additional controller 61/61A/61B/61C (hereinafter, the additional controller 61 or the like) confirms that the emission instruction signal has been turned on, that the image capturing preparation completion signal has been turned on, and that the timer goes over the predetermined waiting time, the system 100A or the like transitions to the emission waiting state St4, as shown in FIG. 15.

In the emission waiting state St4, the additional controller 61 or the like confirms whether the image capturing device 3A/3B is in a state allowed to perform image capturing. The image capturing device 3A/3B confirms whether it is in the state allowed to perform image capturing, and in a case where it is determined as being in the state allowed to perform image capturing, transmits the emission start signal to the additional controller 61 or the like (t5), as shown in FIG. 16 and FIG. 17.

For confirming whether it is in the state allowed to perform image capturing, it is determined whether a predetermined reset operation has been completed, for example, and charges in the light receiver of the image capturing device 3A/3B have been removed, or whether the reset operation has been completed in every pixel on the light receiving surface (because the reset operation is performed by scanning each row of the respective pixels arranged to extend as a matrix on the light receiving surface).

When the additional controller 61 or the like senses that the emission start signal from the image capturing device 3A/3B has been turned on, the system 100A or the like transitions to an emission permission state St5, as shown in FIG. 15.

In the emission permission state St5, the additional controller 61/61A turns on the image capturing start signal which is an internal interlock (t5) as shown in FIG. 16, and turns on the emission permission signal or the emission instruction signal being output to the radiation controller 11/11A, and outputs the timing signal to the image capturing device 3A.

The additional controller 61B/61C turns on the image capturing start signal (t5) to turn on the emission permission signal or the emission instruction signal being output to the radiation controller 11/11A, as shown in FIG. 17.

The radiation generation device (the radiation controller 11/11A, the high voltage generator 12, and the radiation generator 2) generates radiation when the emission permission signal or the emission instruction signal from the additional controller 61 or the like is turned on, which allows radiation passed through the subject to enter the image capturing device 3A/3B.

In the emission permission state St5, control may be exerted to count the number of already captured images each time the additional controller 61 or the like transmits the timing signal after the emission start signal is turned on. In this case, the image capturing start signal is turned off (t6) in a case where the counted number of already captured images reaches the maximum number of captured images N having been set, and the system 100A or the like transitions to an emission termination state St6, as shown in FIG. 15.

In the case of counting the number of already captured images by counting the timing signals, it is necessary to read out a captured image obtained by the last radiation emission. Thus, the timing when turning off the readout instruction signal may be delayed, and a timing signal serving as a trigger of the readout operation may be further transmitted for a frame. Such a configuration enables the danger that image capturing is continued by more than or equal to the maximum number of captured images N having been set, and radiation emission unnecessary for the subject is performed to expose the subject to radiation excessively to be eliminated.

Thereafter, when the radiographer releases the second stage of the emission instruction switch 5, the emission instruction signal is turned off (t7) as shown in FIG. 16 and FIG. 17.

Thereafter, when the radiographer releases the first stage of the emission instruction switch 5, the emission preparation signal is turned off (t8).

When the additional controller 61 or the like confirms that all the signals input thereto have been cancelled, the system 100A or the like transitions to the emission preparation state St2, as shown in FIG. 15.

Herein, “all the signals” include the emission preparation signal, the emission instruction signal, the image capturing start signal which is an interlock of the additional controller 61 or the like, and the emission start signal of the image capturing device 3A/3B.

Thereafter, in a case where the radiographer determines that it is necessary to further perform another type of image capturing, or as a result of confirming a captured image, perform image capturing again because an acquired captured image is insufficient for an intended purpose, changes the state of the subject and image capturing conditions to perform image capturing again in the above-described flow.

In a case where the radiographer determines that it is not necessary to perform image capturing, the console 4 turns off the sequence start signal (t9) to terminate the image capturing sequence. Then, the system 100A or the like transitions to the waiting state St1 as shown in FIG. 15.

The system 100A or the like may transition to the waiting state St1 in a case where there is no input from the radiographer for a certain time, besides the above-described case (the radiographer makes the determination).

Operation When Not Continuing Image Capturing

The flow of the above-described state transition refers to the case in which image capturing is continued up to the maximum number of captured images N, whilst there are cases in which image capturing is not continued up to the maximum number of captured images N depending on various situations.

For example, in a case where the radiographer desires to discontinue image capturing before the maximum number of captured images N are captured, he/she releases the second stage of the emission instruction switch 5 to turn off the emission instruction signal. Then, the system 100A or the like transitions from the emission permission state St5 to the emission termination state St6. This is because one of the plurality of OR conditions (the emission instruction signal from the emission instruction switch 5 is turned off, the emission start signal from the image capturing device 3A/3B is turned off, and the image capturing start signal from the additional device 6 or the like is turned off) for transition from the emission permission state St5 to the emission termination state St6 shown in FIG. 15 has been satisfied.

In the emission termination state St6, radiation emission is stopped, and thereafter, processing of transmitting a remaining image in the image capturing device 3A/3B to the console 4, processing of deleting images stored in the image capturing device 3A/3B after transmission, and the like are performed similarly to the case in which images have been captured up to the maximum number of captured images N. This is for the purpose of, because captured images may be utilized even in a case where images have not been captured up to a previously designated number of images, enabling the radiographer to confirm the captured images even in such a case similarly to normal images.

On the other hand, it is necessary to perform management associating the fact that images have not been captured up to the previously designated number of images with the captured images, and in the case where images have not been captured up to the previously designated number of images, management is performed quoting individual images or a group of images with the fact that images have not been captured up to the previously designated number of images.

In the case where images have not been captured up to the previously designated number of images, the console 4 displays the fact that images have not been captured up to the previously designated number of images by transmitting an error signal from the additional device 6 or the like, for example.

Operations when Error Occurs

There is a case in which connection between the additional device 6 or the like and the image capturing device 3A/3B is disconnected during image capturing. This may be caused by detachment of a cable from a connector in the case where the additional device 6 or the like and the image capturing device 3A/3B are connected by wire, for example, or may be caused by crosstalk, a failure of a wireless device, cut of power to a wireless device, or the like in the case where the additional device 6 or the like and the image capturing device 3A/3B are connected wirelessly.

Therefore, the system 100A or the like may be provided with a function of monitoring whether an error (an error 1, an error 2, an error 3, and an error 4) has occurred in each of the sequence states St3 to St6, and in a case where an error has been sensed, a transition may be made to an error state St7 as shown by broken lines in FIG. 15.

In the case where a transition is made to the error state St7, details of the error having caused the transition to the error state St7 to be made may be displayed on the display 43 of the console 4 or the like.

For such error sensing, an error monitoring sequence for monitoring a signal in each state different from the image capturing sequence as shown in FIG. 15, for example, may be advanced in parallel, and in a case where an error is sensed in the error monitoring sequence, the image capturing sequence may transition from the current sequence states St3 to St6 to the error state St7.

Alternatively, control may be exerted to set an operable time for each of the sequence states St3 to St6 shown in FIG. 15, and start timekeeping with the timer when a transition is made to each of the sequence states St3 to St6 to measure the operation time in each of the sequence states, and in a case where the time of the timer goes over the operable time in the sequence state, make a transition to the error state St7.

Furthermore, in the case where an error has occurred, the console 4 may be notified of the error from the additional device 6 or the like or the image capturing device 3A/3B having sensed the error, and the occurrence of the error may be displayed on the console 4.

After the transition is made to the error state St7, a transition is made to the emission preparation state St2 or the waiting state St1 with satisfaction of a particular condition (cancellation of the error, cancellation of all the signals, or the like) serving as a trigger.

Effects

By using such an error sensing system, a defect of a device or an operation is sensed reliably to cause a transition to be made to the error state, and by making a return to the waiting state St1 or the emission preparation state St2 in the middle of the image capturing sequence according to necessity, the danger that radiation is emitted in a state where the device or operation has the defect to expose the subject to radiation uselessly is eliminated.

### EXAMPLES

Specific examples when implementing the above-described system 100A or the like will now be described.

Various technologies described herein may be applicable to the conventional systems 100, 200, and the like.

### Example 1: Integration of Additional Controller

### Integration of Additional Controller Into Radiation Control Device

When installing the system 100A or the like according to the above-described embodiments in a medical institution such as a hospital, space for providing the additional device 6 or the like independently from the radiation control device 1 is not ensured in some cases depending on the situation of the medical institution.

Some of the radiation control devices 1 have space therein for adding an additional function as an option.

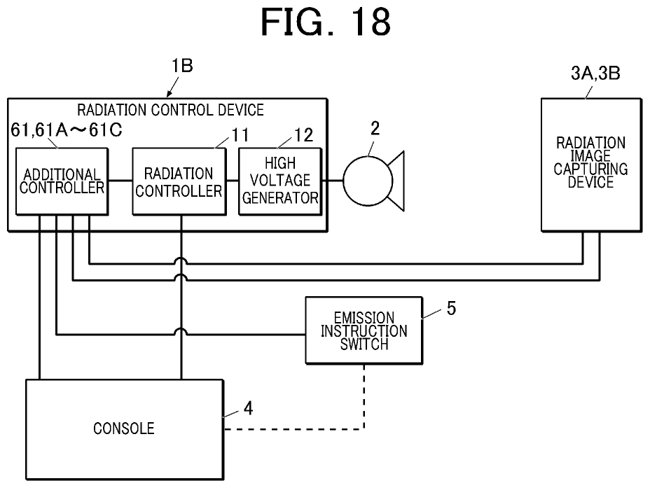

Therefore, the additional controller 61 or the like may be provided inside the radiation control device 1B as shown in FIG. 18, for example, rather than being provided as the additional device 6 or the like independent from the radiation control device 1.

The additional controller 61 or the like is provided in the form of a substrate, for example. Accordingly, by adding the additional controller 61 or the like and, according to necessity, an I/F part to a conventional device that captures still images without providing the additional device 6/6A separately from the radiation control device 1, the system 100A or the like is brought into the state allowed to perform kymography.

In addition, interconnect lines laid out around the respective devices that constitute the system 100A or the like are reduced, which reduces the risk that the interconnect lines interfere with image capturing, or noise received from the interconnect lines causes the system 100A or the like to malfunction.

### Example 2: Medication Cart

### Configuration in Medication Cart

Many radiographers desire to perform kymography not only with a radiography system used in a fixed manner in a room, but also with a medication cart that is moved in a medical institution and used.

Therefore, the configuration of any of the above-described embodiments may be used for a conventional medication cart that captures still images. That is, the additional device 6/6A operates integrally with the medication cart by including the additional device 6/6A in the inner side of an enclosure of the medication cart, or adding the additional device 6/6A to the medication cart so as to be movable with the medication cart.

On that occasion, as described in Example 1 above, the additional controller 61 or the like may be provided in the inner side of the radiation control device 1.

This enables kymography to be performed using the conventional medication cart for capturing still images.

### Example 3: Unification of Display

### Input/Display of Information

In the system 100A or the like according to the above-described embodiments, the radiation control console 41 and the image capturing device control console 42 may each have the display 43. In that case, if different image capturing conditions are displayed on the display 43 of each of the consoles 41 and 42, the radiographer may be unable to discriminate which of the image capturing conditions have been set in the radiation controller 11/11A or the image capturing device 3A/3B, and in the worst case, there is a possibility that image capturing is performed under image capturing conditions not intended by the radiographer to expose a subject to radiation uselessly.

Therefore, control may be exerted to equalize image capturing conditions set in each of the radiation control console 41 and the image capturing device control console 42 to equalize the content displayed on the display 43 of each of them as a consequence. A specific example of control for equalizing the image capturing conditions will be described later in Example 4 below, for example.

Besides the above-described method, triggered by at least one of the fact that at least one of the radiation control console 41 and the image capturing device control console 42 has sensed a particular operation, for example, and the elapse of a particular time, processing of confirming that image capturing conditions set in each of the radiation control console 41 and the image capturing device control console 42 or setting content displayed on the display 43 of each of them agree with each other may be executed.

A result of execution of this processing, that is, at least one of whether the setting content or display content of each of the radiation control console 41 and the image capturing device control console 42 is equal and a warning that the setting content or display content is different between them, may be notified.

Accordingly, also in the case where the radiation control console 41 and the image capturing device control console 42 each have the display 43, equal image capturing conditions are set in each of them, or equal setting content is displayed on each of the displays 43. This enables the radiographer to confirm the image capturing conditions set in the system 100A or the like as a whole, and to perform image capturing under image capturing conditions intended by the radiographer.

### Example 4: Agreement of Input Results

### Information Linking

In the case of enabling image capturing conditions or the like to be input from each of the radiation control console 41 and the image capturing device control console 42 as in the first to fourth embodiments, input results (setting content) in each of the consoles 41 and 42 need to agree with each other.

Therefore, in a case of changing conditions on one of the radiation control console 41 and the image capturing device control console 42, the other console may also be changed to the equal settings by an information linking method as will be described below as (1) to (3), for example.

Information Linking Method (1)

One of the radiation control console 41 and the image capturing device control console 42 is defined as a master, and the other console is defined as a slave. Rewriting of information is performed in the master, and the slave is only allowed to duplicate the information rewritten in the master.

Information Linking Method (2)

The radiation control console 41 and the image capturing device control console 42 both use a common information linking method.

Alternatively, the radiation control console 41 and the image capturing device control console 42 are respectively provided with timers synchronized with each other, and when an input is made, time information of the timers is also stored together with the input content. Then, image capturing conditions are set in chronological order from temporally older inputs in both the radiation control console 41 and the image capturing device control console 42.

Information Linking Method (3)

In a case where an input is made, rewriting is performed in both the radiation control console 41 and the image capturing device control console 42. Until rewriting in both of them is terminated, the next input is not accepted, or the next input is stored, and when rewriting is terminated, rewriting of the next input is performed.

This enables image capturing conditions or the like to be input from either the radiation control console 41 or the image capturing device control console 42, which increases convenience of the radiography system.

In addition, an input made from one of the radiation control console 41 and the image capturing device control console 42 is reliably made agree between both the consoles 41 and 42.

### Example 5: Permit Emission Upon Confirmation That Settings are Equal Confirmation of Information

In the case where conditions such as image capturing conditions are input from both the radiation control console 41 and the image capturing device control console 42, and the image capturing conditions or the like are displayed on both the consoles 41 and 42 as in the first to fourth embodiments, there is a possibility that image capturing is performed under image capturing conditions not intended by the radiographer if image capturing is performed under image capturing conditions set in either the console 41 or 42 in a case where conditions on both the consoles 41 and 42 do not agree with each other.

Therefore, it may be determined at certain timings in the image capturing sequence whether image capturing conditions recognized by, set in, or displayed on the radiation control console 41 and the image capturing device control console 42 agree with each other, and in a case where it is determined that the image capturing conditions agree, the image capturing sequence may be continued.

In the case where the image capturing sequence is continued, the fact that the image capturing conditions agree and there is no problem may be displayed.

In a case where it is determined that image capturing conditions do not agree between the radiation control console 41 and the image capturing device control console 42, at least one of exerting control so as not to permit continuation of the image capturing sequence or emission of radiation and displaying the fact that the image capturing conditions do not agree may be performed.

At least one of the “certain timings in the image capturing sequence” in the above description may be the time when image capturing conditions are set after the emission preparation signal is input, or the time when the confirming operation is performed, as shown in FIG. 16 and FIG. 17, for example.

This enables image capturing conditions to agree reliably between both the radiation control console 41 and the image capturing device control console 42.

The danger that, in the state where the image capturing conditions do not agree, image capturing is performed under image capturing conditions set in either one of the radiation control console 41 and the image capturing device control console 42 is reliably eliminated.

### Example 6: Prevent Image Capturing Conditions From Being Changed After Image Capturing is Started

### Period of Forbidding Information Input/Cange

Even if the radiographer inputs image capturing conditions on one of the radiation control console 41 and the image capturing device control console 42, and image capturing is started in the state where image capturing conditions in the other one of the consoles have been equalized through use of the above-described technology, when a change in the image capturing conditions not intended by the radiographer is made on the radiation control console 41 or the image capturing device control console 42 during subsequent image capturing, image capturing not intended by the radiographer may be performed thereafter.

Therefore, image capturing conditions may be prevented from being changed on the radiation control console 41 and the image capturing device control console 42 subsequent to a certain timing in the image capturing sequence.

Specifically, the display screen is caused to transition to a screen other than the input screen, or the input screen is grayed out or the like to prevent image capturing conditions from being input, for example.

At least one of the “certain timings in the image capturing sequence” in the above description may be the time when image capturing conditions are set after the emission preparation signal is input, or the time of the confirming operation, as shown in FIG. 16 and FIG. 17, for example.

This reliably prevents image capturing conditions from being changed during image capturing to cause image capturing to be performed under image capturing conditions not intended by the radiographer.

### Example 7

When performing image capturing, in which image capturing mode image capturing is to be performed needs to be set previously in the image capturing device 3A/3B and the radiation generation device (the radiation controller 11/11A and the high voltage generator 12).

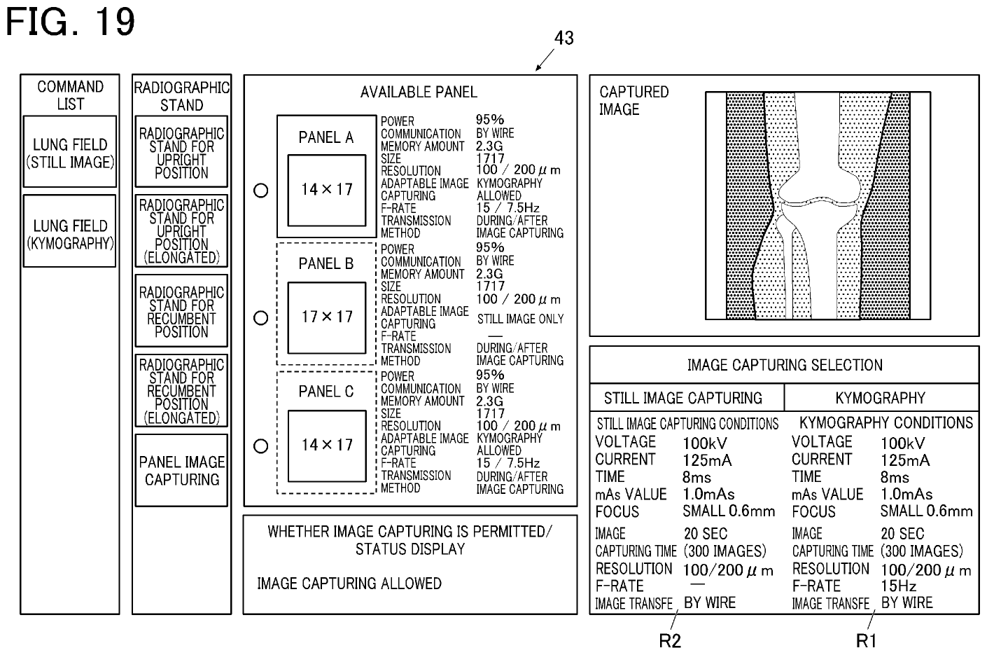

In view of such a problem, after selecting the image capturing device 3A/3B to be used on the console 4 (on the display 43 shown in FIG. 19, for example), an image capturing mode for performing image capturing may be selected on the console 4 from among (1) a still image capturing mode, (2) a continuous image capturing (pulse emission) mode, (3) a continuous image capturing (continuous emission) mode.

Selection and setting of an image capturing mode is performed at any timing after selection of the image capturing device 3A/3B and before image capturing in the image capturing sequence.

On that occasion, depending on the types of the image capturing device 3A/3B and the radiation controller 11/11A, an unadaptable one of the above alternatives may be prevented from being selected on the console 4.

Alternatively, control may be exerted to present an error display in the case where an unadaptable one of the above alternatives is selected, or to prevent image capturing from being performed even if the unadaptable one is selected.

Accordingly, an image capturing mode intended by the radiographer is selected.

An image capturing mode to which the image capturing device 3A/3B and the radiation generation device are not adaptable is prevented from being selected to cause image capturing.

### Example 8

When performing image capturing, it is necessary to set previously whether image capturing is performed in a cooperation state in which the radiation generation device and the image capturing device 3A/3B cooperate with each other, or whether image capturing is performed in a non-cooperation state in which they do not cooperate with each other.

In view of such a problem, after selecting the image capturing device 3A/3B to be used and the image capturing mode on the console 4, a selection may be made as to whether to bring the image capturing device 3A/3B and the radiation generation device into (1) the cooperation state, (2) the non-cooperation state, or (3) the cooperation state until image capturing is started, and after the start of image capturing, the non-cooperation state.

At any timing after the selection and until image capturing is started, the above-described settings will be made in each of the image capturing device 3A/3B and the radiation generation device.

In the case where the cooperation state is selected and set, image capturing in which information about the number of captured images and an image capturing frame rate is shared between the image capturing device 3A and the radiation control device is performed.

In the case where the non-cooperation state is selected and set, the image capturing device 3B will create a timing by itself to perform image capturing.

Accordingly, the frame rate and the number of captured images are transmitted from the console 4 to the image capturing device 3A/3B, and image capturing is performed in a state intended by the radiographer (the cooperation state or non-cooperation state).

A state to which the image capturing device 3A/3B and the radiation generation device are not adaptable is prevented from being selected to perform image capturing.

### Example 9: Output Via Divided Interconnect Lines

### Interconnecting Method

For altering an existing radiography system in order to connect the additional controller 61 or the like, an alteration is made for capturing still images in some cases, and an alteration is made for performing both still image capturing and kymography in other cases.

In the case of making an alteration only for capturing still images, there is no problem because an interconnect line for the timing signal is not necessary, however, when an existing radiographic stand (for upright position, recumbent position, or the like) is used for image capturing, for example, the thickness of interconnect lines arranged in a device to be altered may be restricted to the minimum thickness (for example, the thickness of a generally-used LAN cable or the like) necessary for transmitting/receiving information for capturing still images because of the curvature in the device or the like. When attempting to alter such a device, a problem arises in that the types of signals transmitted/received by way of the inside of the device increase to thicken the interconnect lines, which cannot be arranged in the device.

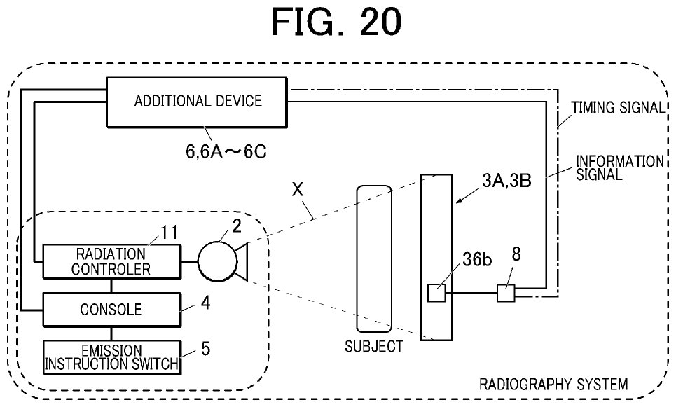

Therefore, output may be made from the additional controller 61 or the like to the image capturing device 3A/3B via signal lines divided into an interconnect line for information, an interconnect line for power feeding, and an interconnect line for timing signal, as shown in FIG. 20, for example.

Specifically, in the case of making an alteration only for capturing still images, the interconnect lines are divided into two (the interconnect line for information and interconnect line for power feeding) and arranged.

In the case of making an alteration for performing both still image capturing and kymography, the interconnect lines are divided into three (the interconnect line for information, interconnect line for power feeding, and interconnect line for timing signal) and arranged.

In the case of making an alteration for performing both still image capturing and kymography, the lines may be divided into two: the interconnect line for information signal and power feeding; and an interconnect line for timing signal, or may be divided into two: the interconnect line for information; and the interconnect line for power feeding and timing signal.

Accordingly, it is not necessary to perform interconnection for the timing signal in the alteration for performing still image capturing, which eliminates the need to pay attention to the thickness of interconnect lines.

In the alteration for performing both still image capturing and kymography, it is necessary to enable the timing signal to be transmitted/received in addition to transmission/reception of information necessary for still image capturing and power feeding. By dividing the interconnect lines into two or more so as not to thicken the interconnect lines, the alteration is performed relatively easily.

### Example 10: Join Respective Interconnect Lines Before Connection

### Joining of Interconnect Lines

The image capturing device 3A/3B is also used in some cases without interconnect lines by transmitting/receiving signals wirelessly, and interconnect lines for information, power feeding, and transfer of the timing signal may be attached/detached by means of a connector or the like.

However, in a case where a plurality of interconnect lines are connected to the connector, a problem arises in that handling of the interconnect lines becomes complicated, and as a result, a necessary interconnect line is disconnected from the connector due to a contact with another interconnect line, for example, to interrupt signal transfer, so that intended image capturing can no longer be performed.

Therefore, in a case where interconnect lines to be connected are divided into each of or any combination of the interconnect line for information, interconnect line for power feeding, and interconnect line for timing signal, at least two or more of these interconnect lines may be joined using a joining device 8 to make the number of interconnect lines smaller than the types of signals to be transmitted/received and to make connection to the image capturing device 3A/3B, as shown in FIG. 20, for example.

Accordingly, the interconnect lines are reduced, and handling of the interconnect lines is simplified, which reduces the risk that intended image capturing can no longer be performed due to detachment of an interconnect line or the like.

The interconnect lines from the additional device 6 or the like to the image capturing device 3A/3B may be divided into an interconnect line required in still image capturing and an interconnect line required in the case of performing kymography in addition to still image capturing.

Examples of this interconnect line required in the case of performing kymography in addition to still image capturing include the interconnect line for transmitting the aforementioned timing signal.

Accordingly, an interconnect line before an alteration can be used as it is as an interconnect line required in still image capturing after the alteration. In the case of performing kymography, a conventional device that only captures still images is easily changed to a device allowed to perform kymography merely by adding an interconnect line required in the case of performing kymography in addition to the interconnect line required in still image capturing existing from before the alteration.

### Example 11: Obtain Information From Radiation Image Capturing Device Via Additional Device

### Connection of Image Capturing Device Via Additional Device

The image capturing device control console 42 in the above-described embodiments is connected to the communication network N, and thus connectable to the other image capturing device 3A not shown by wire or wirelessly via this communication network.

For performing kymography as described above in the first and third embodiments, the image capturing device 3A needs to be connected to the additional controller 61/61A.

Therefore, the image capturing device control console 42 may be caused to perform information communication for performing kymography with the image capturing device 3A via the additional controller 61/61A.

On that occasion, the image capturing device control console 42 is caused to acquire information such as the type of the image capturing device 3A from the image capturing device 3A being connected via the additional controller 61/61A.

Accordingly, it is reliably confirmed in the additional controller 61/61A whether the image capturing device 3A to be used for image capturing is allowed to perform kymography.

### Example 12: Display Whether Kymography is Allowed

### Display of Image Capturing Device Allowed to Perform Kymography

The image capturing device control console 42 in the above-described embodiments is connected to the communication network N, and thus connectable to the other image capturing device 3A/3B not shown by wire or wirelessly via this communication network.

Therefore, as shown in FIG. 19, for example, the radiographer may cause the display 43 of the image capturing device control console 42 to display whether the image capturing device 3A/3B being connected is allowed to capture still images only, or allowed to capture kinetic images in addition to still images.

Displaying whether the image capturing device is allowed to perform kymography on the image capturing device control console 42 enables the radiographer to easily and reliably select the image capturing device 3A/3B allowed to perform kymography.

The radiographer is prevented from selecting an image capturing device not allowed to perform kymography by mistake and performing kymography as it is.

### Example 13: Change Selection Range of Resolution and Frame Rate

### Selection of Conditions for Kymography

The radiographer needs to set an appropriate resolution and frame rate in the image capturing device 3A/3B to be used for image capturing.

Therefore, along with the image capturing device 3A/3B allowed to be used, the resolution and frame rate of that image capturing device 3A/3B may be displayed, as shown in FIG. 19, for example.

A selection may be made from among resolutions and frame rates being displayed.

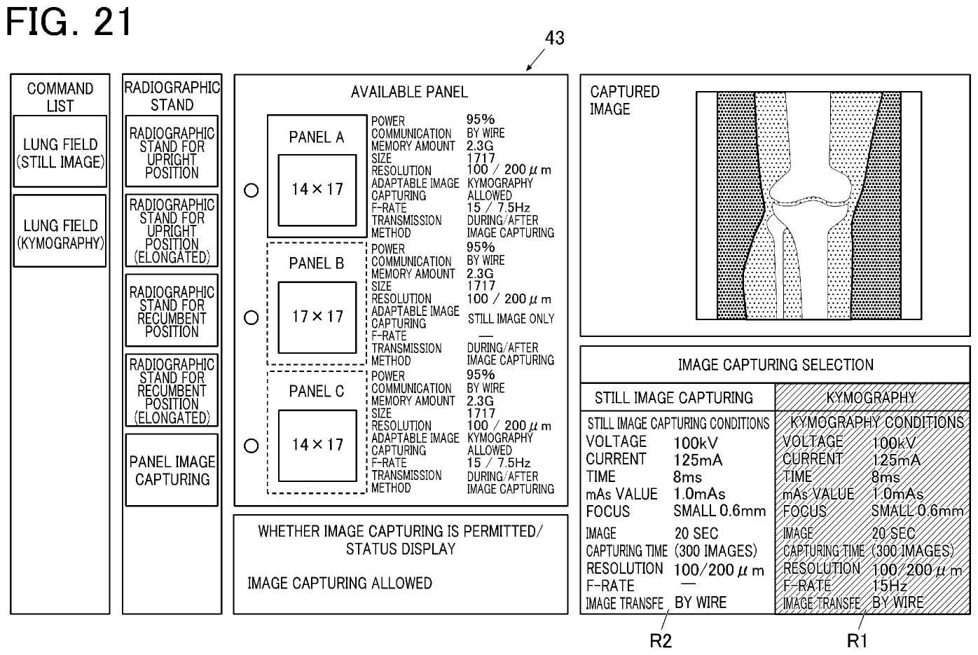

In a case where the image capturing device 3A/3B only adaptable to still image capturing is connected, while displaying it as the image capturing device 3A/3B allowed to be used, a condition setting region R1 of the image capturing device 3A/3B not allowed to be used may be prevented from being selected for kymography by grayout or the like as shown in FIG. 21, for example, may be prevented from being set even if it is selected, or may be prevented from being permitted to perform image capturing.

In addition to the resolution and frame rate, whether binning processing is allowed, an image transmission method, an exposure time, and the like may be displayed. A selection may also be made from among them.

### Example 14: Switch Between Still Image Capturing and Kymography

### Switch Between Still Image Capturing and Kymography

The radiographer timely selects either still image capturing or kymography depending on the situation, and performs image capturing. Therefore, the radiography system needs to be switchable between still image capturing and kymography.

For example, a method of switching a control method between still image capturing and kymography is used. Specifically, when still image capturing is selected, control of the additional controller 61 or the like is switched to perform still image capturing, and when kymography is selected, control of the additional controller 61 or the like is switched to perform kymography.

As shown in FIG. 21, whether still image capturing is selected or kymography is selected currently may be displayed. They may also be switched.

For example, in the case where still image capturing has been selected, the fact that still image capturing has been selected is displayed by graying out the condition setting region R1 for kymography on the display 43, as shown in FIG. 21.

Although illustration is omitted, in a case where the selected image capturing device 3A/3B is the image capturing device 3A/3B adaptable to kymography, the image capturing method is switched to kymography when the grayed-out condition setting region R1 for kymography is selected, and the fact that kymography has been selected is displayed by cancelling grayout of the condition setting region R1, and instead, graying out the condition setting region R2 for still image capturing.

The condition setting region R1/R2 is selected by moving a pointer displayed on the screen to the condition setting region R1/R2 for intended image capturing using a mouse, for example, and clicking the mouse there. Alternatively, in a case where the display 43 is a touch panel screen, the condition setting region R1/R2 for intended image capturing is selected by making a touch.

On that occasion, image capturing conditions may be held for each of still image capturing and kymography, and in the case where still image capturing or kymography is selected, the image capturing conditions for the selected image capturing may be set automatically. The individual image capturing conditions for still image capturing and kymography may be values individually preset in accordance with an image capturing technique, or may be values changed and input by the radiographer.

In this case, in a case where the radiographer selects kymography after setting the image capturing conditions for still image capturing, for example, the image capturing conditions for kymography are displayed and set. Thereafter, in a case where still image capturing is selected again, the image capturing conditions for still image capturing before selecting kymography are set and displayed.

Besides, by setting the maximum number of captured images N at 1 in settings for kymography, control for still image capturing is performed.

Accordingly, even if there is a single radiography system, both still image capturing and kymography are performed in accordance with a selection made by the radiographer.

### Example 15: Reset by Pressing First Stage of Emission Instruction Switch Timing of Starting Reset Operation

Since the reset operation consumes power, consumption power increases if the reset operation is repeated from the start of an image capturing sequence. In particular, in a case of driving the image capturing device 3A/3B with a built-in battery, a problem of battery exhaustion arises.

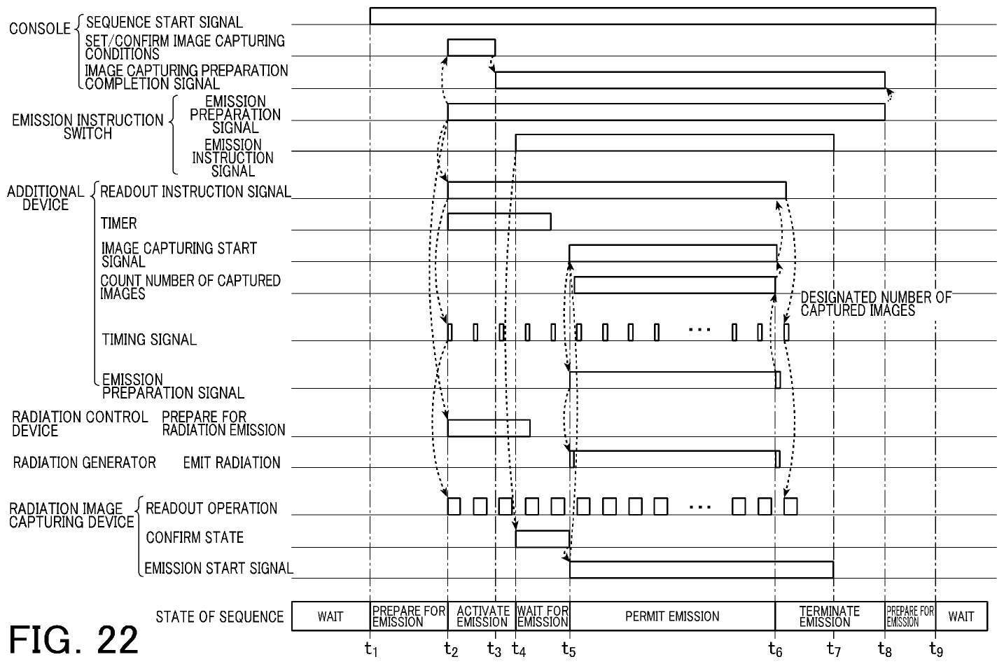

Therefore, the timing of starting the reset operation (the readout operation) may be after the emission preparation signal is turned on, as shown in FIG. 22, for example.

Although illustration is omitted, the timing signal may be output before transmission of the emission preparation signal, and a readout instruction signal different from the timing signal may be transmitted to the image capturing device 3A/3B, and when the image capturing device 3A/3B receives the readout instruction signal, readout may be performed in response to the timing signal.

Accordingly, power consumption of the image capturing device 3A/3B is reduced.

### Example 16: Start Kymography Cycle After Stop of Reset

### Timing of Starting Image Capturing

The reset operation of the image capturing device 3A/3B is performed by scanning each row of respective pixels of a light receiver arranged to extend as a matrix on the surface of a built-in substrate. Thus, if image capturing is started during the reset operation, radiation is received in a state where the reset operation has been completed for a portion of the light receiver, while the reset operation has not been completed for the remaining portion, and the density distribution of a radiation image may vary between the portion for which the reset operation has been completed and the other portion for which the reset operation has not been completed.

Therefore, it is preferable that image capturing is started in a state where the reset operation has been performed uniformly for the whole light receiver of the image capturing device 3A/3B.

Specifically, the image capturing device 3A/3B is provided with a function of transmitting the emission start signal to the additional device 6/6A at a timing when scanning of all the pixels of the light receiver is completed after the start of the reset operation.

Then, the additional device 6/6A receives the emission start signal at the timing when the reset operation is performed uniformly, and turns on the image capturing start signal to be an interlock to repeatedly transmit the emission permission signal to the radiation controller 11/11A.

This reliably prevents image capturing from being performed in a state where the reset operation for the light receiver is not uniform to cause the density distribution of a radiation image to vary between the portion for which the reset operation has been completed and the other portion for which the reset operation has been completed.

### Example 17: Discriminate Whether Emission State has Been Brought About Based on Difference in Timing Signal

### Signal When Radiation is Not Emitted/When Radiation is Emitted

Merely with the timing signal, the image capturing device 3A/3B is not capable of discriminating whether the readout operation is being performed as the reset operation (while radiation is not being emitted), or performed as image capturing (while radiation is being emitted). Thus, whether to store a read-out image as a captured image is discriminated.

Therefore, the timing signal for the reset operation and the timing signal for image capturing may be made different from each other.

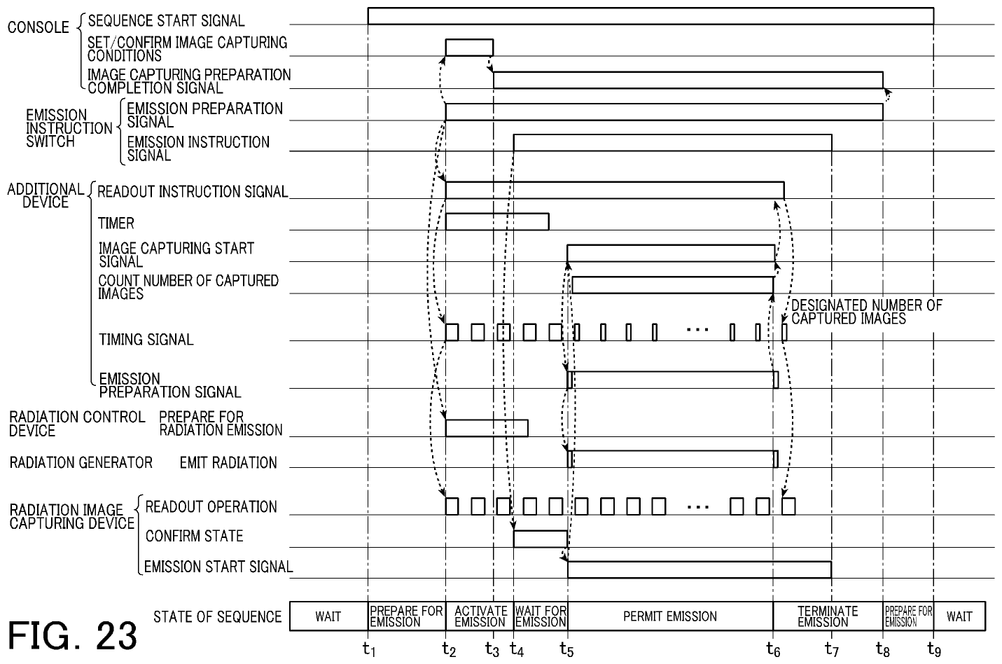

Specifically, as shown in FIG. 23, for example, there is a method such as changing the signal pulse width between the timing signal for the reset operation and the timing signal for image capturing. Even if the signal pulse width is changed, the timings of the reset operation and image capturing will not be mismatched if the readout operation is started in response to the rising of a pulse signal.

This enables the image capturing device 3A/3B to discriminate whether the readout operation is being performed as the reset operation or as image capturing based on the timing signal. Thus, the danger that, even though the timing signal is received while radiation is being emitted, it is determined by mistake that the readout operation to be performed is the reset operation to discard a read-out captured image is reliably eliminated

### Example 18: Include Waiting Time Upon Receipt of Emission Preparation Signal

### Waiting Time

Depending on the radiographer, an interval between pressing of the first stage of the emission instruction switch 5 (emission preparation signal output) and pressing of the second stage (emission instruction signal output) may be short. Depending on the device configuration, signals output from the emission instruction switch 5 are not divided into the emission preparation signal and the emission instruction signal, and the emission preparation signal and the emission instruction signal may be input as identical signals. Then, there is a possibility that kymography is started in a state where preparation for image capturing, such as rotation of the rotary anode, warm-up of the image capturing device 3A/3B achieved by the reset operation of the image capturing device 3A/3B, and image uniformization, is insufficient.

This may not arise any problem when capturing a single still image. In the case of kymography, however, an obtained kinetic image may be analyzed using a signal value difference between a plurality of images whose image capturing times are continuous or the like, and a problem may arise in that the frame changes along with changes in the state of the image capturing device 3A/3B during kymography.

Therefore, the additional device 6 or the like may be provided with a timer, and timekeeping with the timer may be started when the emission preparation signal is received, and the emission permission signal may be prevented from being output until a predetermined waiting time elapses after the start of timekeeping with the timer, even if the emission instruction signal is input.

This enables the reset operation of the image capturing device 3A/3B or the like to be performed sufficiently during the waiting time. Since the degree of changes in temperature of the image capturing device 3A/3B after warm-up is performed sufficiently is reduced, a frame change due to a state change of the image capturing device 3A/3B during kymography is prevented.

### Example 19: Make Waiting Time of Additional Device Longer Than Waiting Time of Radiation Controller

### Setting of Waiting Time

Some radiation controllers 11/11A may have a function of not transmitting the emission signal until a predetermined waiting time elapses in a case where the interval between the time at which the emission preparation signal is received and the time at which the emission instruction signal is received is short.

In a case where such a radiation controller 11/11A and the additional device 6 or the like having the function as described in Example 18 above constitute the system 100A or the like, the waiting time to be set in the additional device 6 or the like may be made longer than the waiting time to be set in the radiation controller 11/11A.

This enables a waiting time obtained by adding the waiting time necessary for the image capturing device 3A/3B and the waiting time necessary for radiation emission to be set.

The waiting time to be set in the radiation controller 11/11A may be made zero, and a necessary waiting time may be delayed by the additional device 6 or the like.

In a case of setting the waiting time in the radiation controller 11/11A, the setting is made only in consideration of a delay of radiation. However, this enables a waiting time obtained by adding the waiting time necessary for the image capturing device 3A/3B and the waiting time necessary for radiation emission to be set by setting for the additional controller 61 or the like.

### Example 20: Include Waiting Time Only in Capturing of First Image

### Timing of Waiting Time

In kymography, the waiting time is necessary at the start-up of the device. The waiting time is necessary only for capturing a first image, and the waiting time does not need to be included in capturing of the second and subsequent images.

Therefore, the waiting time may be included only in capturing of the first image, and may not be included in capturing of the second and subsequent images.

This enables the waiting time to be set in the image capturing device 3A/3B and radiation emission only in capturing of the first image which requires the waiting time.

### Example 21

The start of accumulation of charges may be triggered by the fact that the radiation detecting element 32d in the leading (topmost) row in which readout is to be performed first among the radiation detecting elements 32d provided in a plurality of rows has sensed radiation emission.

Since the image capturing device 3B reads out and images a radiation image from each of the radiation detecting elements 32d in each row, images read out from the leading row are generated by exerting control such that image generation is started at a timing when the first or proximate radiation detecting element 32d in a certain row senses radiation emission.

### Example 22

The start of image generation may be triggered by the fact that the radiation detecting element 32d in a row other than the leading row has sensed radiation emission.

Accordingly, even in a case where a time is required from radiation sensing to the start of image generation, radiation is emitted until readout in the leading row is performed next, and thus, images read out from the leading row are generated.

### Example 23

The start of emission of radiation may be determined using sensed data obtained from the plurality of radiation detecting elements 32d, respectively. For example, radiation sensing may be determined based on an average value or the like of a plurality of pieces of sensed data in order to increase noise immunity

This enables emission of radiation to be sensed more correctly.

### Example 24

Radiation may be sensed with the radiation detecting elements 32d of the image capturing device 3B or a radiation sensor provided separately from the radiation detecting elements 32d.

This eliminates the need to use a new component, and simplifies the device configuration.

The use of the radiation sensor enables small-dose pulsed radiation which is difficult to detect with the radiation detecting elements 32d to be reliably detected.

### Example 25

Sensing of radiation with the radiation detecting elements 32d or the radiation sensor may be continued while accumulation of charges and readout of an image are being repeated, and accumulation of charges and readout of an image may be terminated when radiation is no longer sensed.

Herein, “radiation is no longer sensed” refers to a case in which a measured value of radiation becomes less than or equal to a predetermined threshold value.

This prevents image capturing from being continued uselessly even though radiation is not emitted.

### Example 26

The image capturing device 3B triggered by radiation sensing to start image capturing as in the second and fourth embodiments may generate a signal similar to radiation sensing because of changes in load state acting on itself. Thus, a problem arises in that the image capturing device 3B incorrectly determines that radiation has been sensed at an incorrect timing to start image capturing.

Since wireless communication performed between the additional device 6C and the image capturing device 3B uses a best-effort packet transmission technology, the time at which a signal arrives varies, so that it is difficult to use wireless communication for controlling the image capturing timing.

In view of such a problem, the additional device 6A/6C may output an accumulation start signal to the image capturing device 3B, and the image capturing device 3B may be triggered by the fact that the accumulation start signal input from the additional device 6A/6C is turned on to start accumulation of charges and readout of an image.

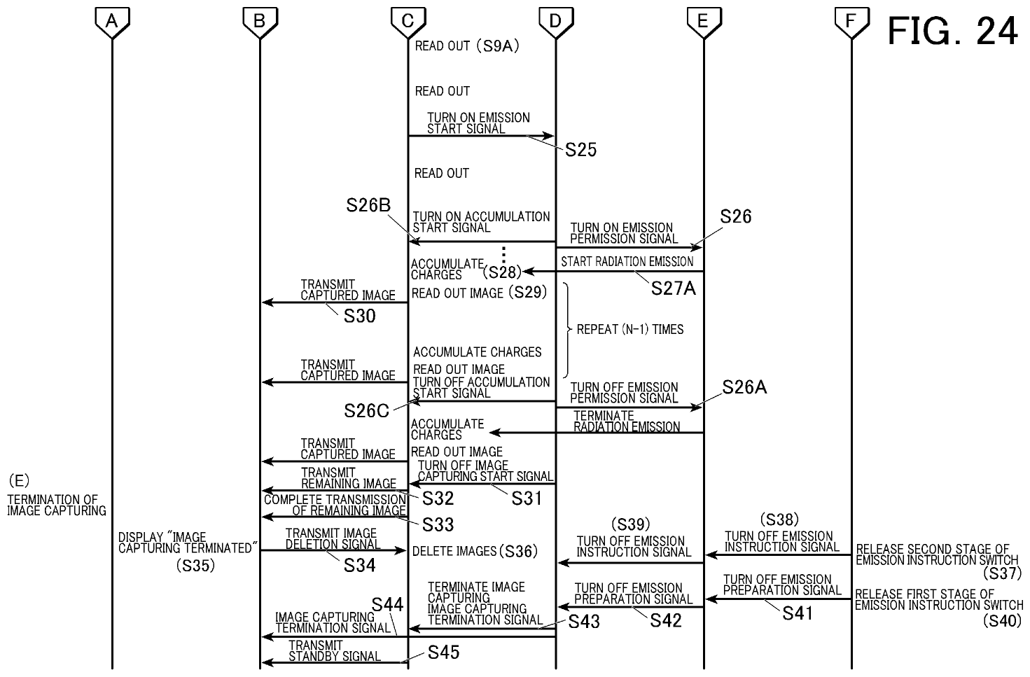

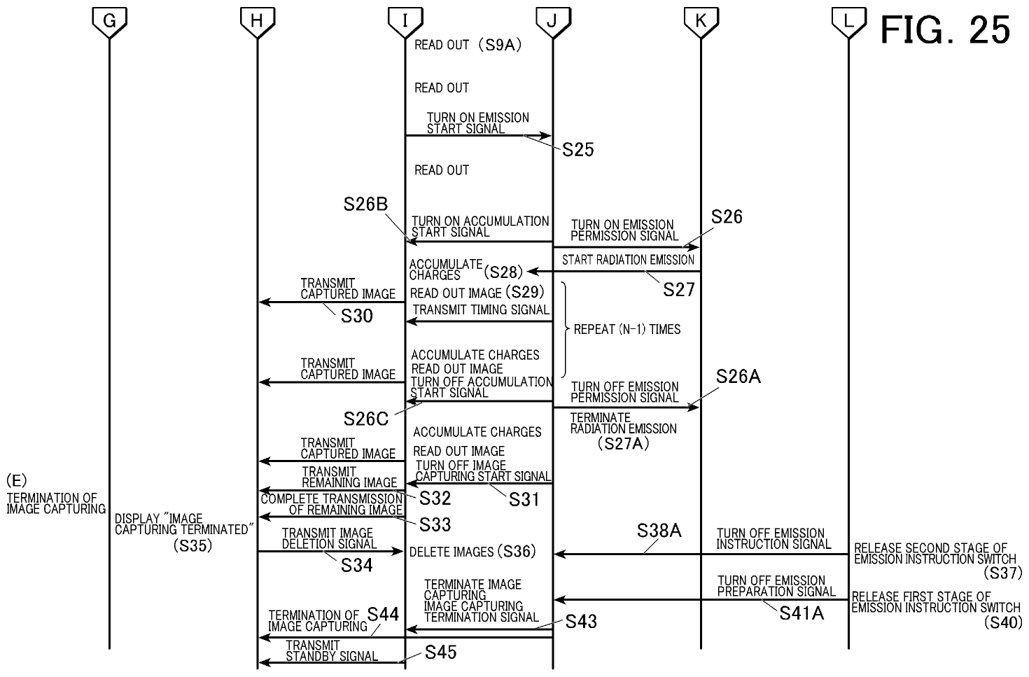

In such a case, the system 100B operates such that the additional device 6A/6C turns on the accumulation start signal to be output to the image capturing device 3B after step S25 (turn on emission start signal) (step S26B), and the image capturing device 3B starts step S28 (accumulate charges) and step S29 (read out image), as shown in FIG. 24 and FIG. 25, for example.

The system 100B also operates such that, when the additional device 6A/6C turns off the accumulation start signal (step S26C) after the image capturing device 3B repeats step S28 and step S29 N times, step S28 and step S29 are terminated.

A method of transmitting the image capturing start signal through wireless communication at a timing slightly before radiation emission by adding signal delay in wireless communication can also be used.

A signal communication system that has no delay or compensates for delay such that signal delay in wireless communication does not occur may be used to transmit the image capturing start timing.

This prevents image capturing from being incorrectly started determining that radiation has been sensed at an incorrect timing.

### Example 27

In a case where the image capturing device 3B fails to start image capturing for such a reason that the accumulation start signal has not been arrived at the image capturing device 3B, a problem arises in that the radiation generation device does not recognize whether image capturing is performed on the image capturing device 3B side, and thus continues emitting radiation to expose a subject to radiation uselessly.

In view of such a problem, the image capturing device 3B may be provided with a function of, in a case where the accumulation start signal is turned on and accumulation of charges and image generation are started, replying that image capturing has been started to the radiation generation device and the additional device 6A/6C. The radiation generation device or the additional device 6A/6C may be provided with a function of determining that image capturing has failed in a case where there is no reply within a predetermined period after the emission start signal is turned on, and stopping radiation emission.

Even in a case of automatically starting accumulation of charges and image generation without using the accumulation start signal as in the above-described embodiments, similarly, the start of image capturing may be returned to the radiation generation device and the additional device 6A/6C after radiation is sensed.

This enables the radiation generation device side to confirm that the image capturing device 3B side reliably performs image capturing to continue image capturing. As a result, emission of radiation is reliably prevented from being continued uselessly in the state where the image capturing device 3B side is not allowed to perform image capturing to expose the subject to radiation uselessly.

### Example 28

In a case where the image capturing device 3B side and the radiation generation device side use different timers independent from each other to synchronize operations during image capturing, a problem arises in that, as the difference in counting between the respective timers increases, the timing for radiation emission by the radiation generation device and the timing for image generation by the image capturing device 3B are mismatched, so that an intended image is not obtained.

In view of such a problem, the image capturing device 3B and the radiation generation device may be connected by wire only immediately before image capturing to synchronize the respective timers, or a TSF function may be used to periodically synchronize the timings.

Accordingly, even in the case where the image capturing device 3B performs image generation at a timing based on the timer independent from the radiation generation device side, the image capturing device 3B is operated in synchronization with the radiation emission timing on the radiation generation device side, which enables more correct image capturing.

### Example 29: Terminate Image Capturing at Set Maximum Number of Captured Images

### Stop of Image Capturing at Designated Number of Captured Images

When image capturing is performed to capture images more than or equal to the maximum number of captured images N previously set even if the radiation emission instruction (pressing of the second stage of the emission instruction switch 5) is continued by the radiographer, a problem arises in that a subject is exposed to radiation uselessly.

Therefore, at least one of the additional controller 61 or the like, the image capturing device 3A/3B, and the radiation controller 11/11A may be provided with a function of counting the number of already captured images.

Furthermore, at least one of the additional controller 61 or the like, the image capturing device 3A/3B, and the radiation controller 11/11A (which may or may not be the same as the one provided with the counting function) is provided with a function of comparing the counted number of already captured images with the maximum number of captured images N previously set, and in a case where the maximum number of captured images N has been reached, transmitting the fact that the maximum number of captured images N has been reached to at least one of the additional controller 61 or the like, the image capturing device 3A/3B, and the radiation controller 11/11A (including transmission from its own controller to its own controller).

The additional controller 61 or the like is provided with a function of, in a case where the fact that the maximum number of captured images N has been reached is received, turning off the image capturing permission signal and stopping output of the emission permission signal.

It is preferable that the readout instruction or the timing signal for readout is output further for capturing at least a single image even after the maximum number of captured images N is reached. This is because, in a case where radiation emission is counted, an image on which radiation emission is performed at the end needs to be read out and stored. In a case where readout completion is counted, this function is not necessary.

Accordingly, image capturing is reliably terminated when capturing of a necessary number of images is completed, which prevents the subject from being exposed to radiation uselessly.

### Example 30

On the radiation generation device side, the number of captured images may be counted, or the image capturing time calculated based on the number of captured images and frame rate may be counted, and determining that image capturing is to be terminated when the number of captured images or the image capturing time reaches a predetermined value, radiation emission may be terminated.

On the image capturing device 3B side, the number of captured images may be counted, or the image capturing time calculated based on the number of captured images and frame rate may be counted, and determining that image capturing is to be terminated when the number of captured images or the image capturing time reaches a predetermined value, the image capturing termination signal may be transmitted from the image capturing device 3B to the radiation generation device to terminate radiation emission.

On that occasion, the number of captured images may be set such that image capturing is performed for a period longer than the radiation emission period.

Accordingly, termination of radiation emission is determined independently from the image capturing device 3B to terminate radiation emission, which enables kymography to be terminated without close cooperation with the image capturing device 3B.

### Example 31

On the image capturing device 3B side, the number of captured images may be counted, or the image capturing time calculated based on the number of captured images and frame rate may be counted, and determining that image capturing is to be terminated when the number of captured images or the image capturing time reaches a predetermined value, image generation may be terminated.

On the radiation generation device side, the number of captured images may be counted, or the image capturing time calculated based on the number of captured images and frame rate may be counted, and determining that image capturing is to be terminated when the number of captured images or the image capturing time reaches a predetermined value, the image capturing termination signal may be transmitted from the radiation generation device side to the image capturing device 3B to terminate image generation.

On that occasion, the number of captured images may be set such that image capturing is performed for a period longer than the radiation emission period.

Accordingly, termination of radiation emission is also determined independently from the image capturing device 3B to terminate radiation emission, which enables kymography to be terminated without close cooperation with the image capturing device 3B.

### Example 32: Confirm Emission Instruction, Number of Already Captured Images, and Presence/Absence of Abnormality, and Continue Image Capturing

### Termination of Image Capturing

A problem arises in that, when image capturing is continued in a case where the radiation emission instruction is interrupted, in a case where capturing of the maximum number of captured images N previously set is finished, in a case where there is a defect in devices that constitute the radiography system or a control state of them, or the like, image capturing is continued in a state not intended by the radiographer to cause the subject to be exposed to radiation uselessly.

Therefore, at least one of whether the radiation emission instruction (pressing of the second stage of the emission instruction switch 5) is being continued, whether the number of already captured images is less than or equal to the maximum number of captured images N previously set, and whether there is a defect in devices or control state may be monitored, and in a case where it is determined that there is a problem, at least one of interruption of image capturing, transmission of the fact that there is a problem, and displays that there is a problem may be carried out.

Such a monitoring operation may be carried out using control for causing the sequence state to transition as shown in FIG. 15, for example.

A monitoring sequence other than in this control may be operated at the same time, and may be monitored. In this case, double-check is made, which enables the occurrence of a problem to be detected more reliably.

This prevents image capturing from being performed in a state not intended by the radiographer to cause the subject to be exposed to radiation uselessly.

The above-described monitoring operation may be performed before initial transmission of the emission permission signal, or before transmission of each of emission permission signals repeatedly performed.

This enables a determination to be made as to whether there is an abnormality for a state immediately before transmitting the emission permission signal that permits emission of radiation, which prevents radiation from being emitted (image capturing from being performed) in a state not intended by the radiographer.

### Example 33

In a case of performing image capturing in the non-cooperation mode in which the radiation generation device and the image capturing device 3B side do not cooperate with each other, a problem arises in that, in a case where a problem arises on the radiation generation device side, and emission of radiation can no longer be continued or the timing is mismatched, the image capturing device 3B fails to recognize the problem on the radiation generation device side, and thus continues image capturing as it is.

In view of such a problem, in a case where there is an abnormality on the radiation generation device side to stop radiation emission, stop of radiation emission may be notified from the radiation generation device to the image capturing device 3B even in the non-cooperation mode to stop image generation performed by the image capturing device 3B.

Accordingly, in a case where a problem arises in the radiation generation device even in image capturing in the non-cooperation mode in which the radiation generation device and the image capturing device 3B do not cooperate with each other, image generation performed by the image capturing device 3B is stopped appropriately, which prevents useless image capturing from being performed.

### Example 34

In the case of performing image capturing in the non-cooperation mode in which the radiation generation device and the image capturing device 3B side do not cooperate with each other, a problem arises in that, in a case where a problem arises on the image capturing device 3B side, and image generation can no longer be continued or the timing of image generation is mismatched, the radiation generation device fails to recognize the problem on the image capturing device 3B side, and thus continues radiation emission even though the image capturing device 3B is in a state where image generation can no longer be continued, which causes the subject to be exposed to radiation uselessly.

In view of such a problem, in the case where there is an abnormality on the image capturing device 3B side to stop image generation, stop of image generation may be notified from the image capturing device 3B to the radiation generation device even in the non-cooperation mode to stop radiation emission performed by the radiation generation device.

Accordingly, in a case where a problem arises on the image capturing device 3B side even in image capturing in the non-cooperation mode in which the radiation generation device side and the image capturing device 3B side do not cooperate with each other, radiation emission performed by the radiation generation device is stopped appropriately, which prevents useless image capturing from being performed, and also prevents the subject from being exposed to radiation uselessly.

### Example 35

In moving image capturing, many images need to be transmitted, which places a load on the network. In particular, in image capturing in the non-cooperation mode of capturing images without causing the radiation generation device and the image capturing device 3B to cooperate with each other as in the second and fourth embodiments, the radiation generation device and the image capturing device 3A/3B may be connected wirelessly to perform communication. In such a case, a further load is placed on the network.

In view of such a problem, images may be transmitted after image capturing is terminated without transmitting images during image capturing.

This enables the load placed on the network to be reduced.

### Example 36

In view of the problem that moving image capturing places a load on the network, and a further load may be placed on the network in the case of the non-cooperation mode, as described in Example 35 above, preview images of all the frames may be transmitted first, and thereafter full images of all the frames may be transmitted.

This also enables the load placed on the network to be reduced.

### Example 37

In view of the problem that moving image capturing places a load on the network, and a further load may be placed on the network in the case of the non-cooperation mode, as described in Example 35 above, kymography in a memory image capturing mode of storing images in the image capturing device 3B during image capturing based on a predetermined manipulation on the console 4 or the image capturing device 3B may be started.

On that occasion, the number of captured images and frame rate which are parameters on the image capturing device 3B side in kymography may be changed by a manipulation of the radiographer.

The parameters may be displayed on the display of the console 4 or the image capturing device 3A/3B.

This also enables the load placed on the network to be reduced.

### Example 38

In view of the problem that moving image capturing places a load on the network, and a further load may be placed on the network in the case of the non-cooperation mode, as described in Example 35 above, images may be stored in the image capturing device 3B during image capturing in the case of image capturing in the non-cooperation mode.

This also enables the load placed on the network to be reduced.

### Example 39

In view of the problem that moving image capturing places a load on the network, and a further load may be placed on the network in the case of the non-cooperation mode, as described in Example 35 above, preview images may be transmitted wirelessly, and full images may be transmitted later after a wired connection is made.

This also enables the load placed on the network to be reduced.

Obviously, the present invention is not limited to the above-described embodiments and the like, and may be changed as appropriate within the scope of the present invention.

For example, a radiation generation device already introduced in a medical institution that emits radiation only once in response to an instruction to emit radiation once is also altered to be allowed to perform kymography by virtue of the technologies described in the present invention.

Alternatively, a novel system allowed to perform kymography is easily established by combining the radiation generation device that emits radiation only once in response to an instruction to emit radiation once and the technologies described in the present invention.

Although embodiments of the present invention have been described and illustrated in detail, the disclosed embodiments are made for purposes of illustration and example only and not limitation. The scope of the present invention should be interpreted by terms of the appended claims

The entire disclosure of Japanese patent application No. 2019-142716, filed on Aug. 2, 2019, is incorporated herein by reference in its entirety.

What is claimed is:

1. A radiation generation control device comprising:
an acquisitor that acquires a first signal that instructs emission of radiation;
a first connector that inputs a second signal indicating a driving state of a radiation image capturing device that generates a radiation image;
a second connector connectable to a radiation generation device that generates radiation; and
a controller that, based on the first signal having been acquired and the second signal having been input, continuously causes a third signal that instructs emission of radiation to be output from the second connector for a predetermined period, wherein
the controller determines the length of the predetermined period in accordance with an image capturing time or the number of captured images previously set.

2. The radiation generation control device according to claim 1, wherein the second connector is a connector in which one end of a cable having the other end connected to the radiation generation device is to be inserted.

3. The radiation generation control device according to claim 1, wherein the second connector is to be connected to the radiation generation device via a relay that relays a signal.

4. The radiation generation control device according to claim 1, wherein
an emission instruction switch that outputs the first signal is connected, and
the acquisitor directly acquires the first signal from the emission instruction switch.

5. The radiation generation control device according to claim 1, wherein
a substrate or an apparatus provided with an emission instruction switch that outputs the first signal is connected, and
the acquisitor acquires the first signal output from the emission instruction switch via the substrate or the apparatus.

6. The radiation generation control device according to claim 1, wherein the acquisitor acquires the first signal output from the emission instruction switch via the radiation generation device.

7. The radiation generation control device according to claim 1, wherein
the controller causes the first connector to output a fourth signal that instructs a timing of capturing the radiation image,
the acquisitor acquires a fifth signal that instructs start of capturing a radiation image, the fifth signal being output before the first signal, and
the controller outputs the fourth signal after acquiring the fifth signal and before acquiring the first signal.

8. The radiation generation control device according to claim 7, wherein
the acquisitor acquires a sixth signal that instructs preparation for radiation emission, the sixth signal being output before the first signal and after the fifth signal, and
the controller outputs the fourth signal after acquiring the sixth signal and before acquiring the first signal.

9. The radiation generation control device according to claim 7, wherein the controller does not output the third signal even if the second signal is input, after acquiring the first signal and before a predetermined waiting time elapses.

10. The radiation generation control device according to claim 7, wherein the controller continues causing the third signal to be output until a predetermined output time elapses after the third signal is output first, or until the fourth signal reaches a predetermined number of times of output.

11. A radiation generation control system comprising:
the radiation generation control device as defined in claim 7; and
a console that is connected to the radiation generation control device, and sets an operation of the radiation generation control device, wherein
the console sets the predetermined period or the number of times of outputting the fourth signal in the radiation generation control device before the radiation generation control device outputs the third signal.

12. The radiation generation control system according to claim 11, wherein the console has a display, and
displays the predetermined period or the number of times of outputting the fourth signal set in the radiation generation control device on the display.

13. The radiation generation control system according to claim 12, wherein the console displays a fact that emission is allowed on the display when the second signal is input to the first connector of the radiation generation control device.

14. The radiation generation control system according to claim 12, wherein the console displays a fact that radiation is being emitted on the display while the radiation generation control device is outputting the third signal.

15. A radiography system comprising:
a radiation generation device that generates radiation;
a radiation image capturing device that generates a radiation image; and
the radiation generation control device as defined in claim 1.

16. A radiography system comprising:
a radiation generation device that generates radiation;
a radiation image capturing device that generates a radiation image; and
the radiation generation control system as defined in claim 11.

17. The radiography system according to claim 15, wherein still image capturing is performed in a state where the radiation generation control device is not connected.
